(19) United States
(12) Patent Application Publication
(10) Pub. No.: US 2013/0295100 A1
(43) Pub. Date: Nov. 7, 2013

(54) MARKERS FOR ACUTE KIDNEY INJURY AND USES THEREOF

(71) Applicants: Paolo Manunta, Milano (MI) (IT); Lorena Citterio, Milano (MI) (IT); Giovanni Guglielmo Landoni, Milano (MI) (IT); Elena Giovanna Bignami, Milano (MI) (IT); Elisabetta Messaggio, Milano (MI) (IT)

(72) Inventors: Paolo Manunta, Milano (MI) (IT); Lorena Citterio, Milano (MI) (IT); Giovanni Guglielmo Landoni, Milano (MI) (IT); Elena Giovanna Bignami, Milano (MI) (IT); Elisabetta Messaggio, Milano (MI) (IT)

(73) Assignee: OSPEDALE SAN RAFFAELE S.R.L., Milano (MI) (IT)

(21) Appl. No.: 13/988,437

(22) PCT Filed: Nov. 21, 2011
(86) PCT No.: PCT/EP2011/070540

§ 371 Date: Jun. 26, 2013

Related U.S. Application Data
(60) Provisional application No. 61/415,431, filed on Nov. 19, 2010.

(51) Int. Cl. C12Q 1/68

(52) U.S. Cl. CPC C12Q 1/6883
USPC 424/137.1; 435/6.11; 549/505; 530/387.5; 514/172

(57) ABSTRACT

The present invention relates to a method for predicting the risk of developing acute kidney injury in a subject from which a biological sample is obtained comprising: detecting the presence of at least one genomic single nucleotide polymorphism (SNP) selected from the group of: ADD1 rs4961 Trp [allelic genotype GT or TT], ADD2 rs4984 [allelic genotype CT or TT], HS-D3B1 rs2236780 [allelic genotype GG], LSS rs914247 [allelic genotype AA], MDR1 rs1045642 [allelic genotype TC or CC], SLC8A1 rs1 1893826 [allelic genotype AA], TRPC6 rs7925662 [allelic genotype CC] from said biological sample, wherein the presence of said allelic genotype is predictive of the risk of developing acute kidney injury.

### FIELD OF THE INVENTION

The present invention relates to the predictive role of plasma endogenous ouabain (EO) levels and genetic single nucleotide polymorphisms (SNPs) in genes involved in EO synthesis (Lanosterol Synthase, LSS, 3β-hydroxyl-steroid dehydrogenase HSD), in EO metabolism (P-glycoprotein encoded by MDR1) and in EO activity (Na/Ca exchanger, SLC8A1, C-type transient receptor potential TRPC6, and adducins, ADD1, ADD2 and ADD3), for assessing the risk of developing severe renal dysfunctions.

### BACKGROUND ART

Endogenous Ouabain (EO) is a cardiac glycoside, structurally similar to digoxin, secreted by adrenal glands.¹ It modulates the activity of the membrane bound Na⁺/K⁺-ATPase pump and induces signal transduction via a second messenger system. Na⁺/K⁺-ATPase modulation in the vascular bed decreases vascular compliance by increasing smooth muscle tone. EO exerts a biphasic effect on Na⁺/K⁺-ATPase, either stimulating or inhibiting its activity, as function of low (subnanomolar) or high (nanomolar) concentrations.^{2,3} EO activated Na⁺/K⁺-ATPase generates Src-kinase and promotes cardiomyocyte hypertrophy and kidney damage. Gene variants involving EO metabolism may modulate this signaling by changing EO tissue levels.⁴

Circulating EO levels have been found increased in hypertension,⁵ and cardiomyopathy.⁶ In particular, in never treated hypertensive subjects plasma creatinine increases and glomerular filtration rate (GFR) decreases across the EO quartile,⁷ suggesting a direct relationship between a progressive impairment of renal function and a chronic exposure to elevated EO plasma levels.

Two mechanisms, among others, are involved in determining the abnormalities of tubular Na⁺ reabsorption observed in essential hypertension: the polymorphism of the cytoskeletal protein alpha-adducin and the increased circulating levels of endogenous ouabain (EO).^{8,9} Both of them lead to increased activity and expression of the renal Na⁺/K⁺ pump, the driving force for tubular Na⁺ transport. Morphological and functional cardiovascular alterations have also been associated with adducin and EO. Rostafuroxin is a new oral antihypertensive agent able to selectively antagonize adducin and EO hypertensive and molecular effects.

Patent application WO2007/060206 discloses Rostafuroxin as a prototype drug able to interfere with the cellular and molecular alterations caused by the mutated adducin and/or increased EO, namely upregulation of the renal Na⁺/K⁺ pump, thus representing the appropriate hypertensive therapy for patients in which these mechanisms are at work. U.S. Pat. No. 5,844,091 discloses methods for diagnosing and monitoring pre-hypertension, hypertension, congestive cardiomyopathy, renal failure, salt-sensitivity and adenomas and endocrine cell hyperplasias by using an antibody having binding specificity to ouabain. Patent application WO2001/025281 relates to a monoclonal antibody or antigen binding fragment thereof having binding specificity for ouabain and to diagnostic and therapeutic uses thereof.

In untreated hypertensive subjects, plasma creatinine increases and glomerular filtration rate decreases across the EO quartile, suggesting a direct relationship between a progressive impairment of renal function and a chronic exposure to elevated EO plasma levels.⁷

In order to corroborate the in vivo adverse consequences of high EO concentration, experimental data on animal models of ouabain-infused rats developing renal dysfunctions have been reported. Enhanced vasoconstriction has been observed in both renal interlobar arteries and descending vasa recta using nanomolar concentration of ouabain and prolonged treatment upregulates the Na⁺ pump α₂-subunit-NCX1-TRPC6 Ca²⁺⁺ signaling pathway in arterial myocytes in vitro as well as in vivo. Therefore, these data suggest the further role of ouabain directly involved as hypertensive factor.^{5,7}

EO concentration, and especially its increase in hypertensives, is known to be related to hemodynamic stress. Similar to aldosterone and cortisol, secretion of EO from adrenocortical cells increases in response to adrenocorticotropic hormone (ACTH).¹⁰

In particular, rapid EO changes have been previously described in literature, in athletes after 15 minutes of ergometry exercise. The increase in EO concentration and its adrenal secretion is partially under the control of norepinephrine and angiotensin II. This suggests the relationship between EO and both lactate and norepinephrine, and explains the finding that EO release is inhibited by beta blockade.¹¹

Therefore, EO is a stress hormone secreted by the adrenal gland and in ill patients is elevated to compensate for hemodynamic dysfunction.

Post-operatively, Acute Kidney Injury (AKI) is an important cause of adverse events within 30 days. Prognostic renal stratification clinical scores based on clinical characteristics (age, sex, ejection fraction etc.)¹²⁻¹⁵ are used to predict AKI and identify patients who are at a greater risk for developing such injury. However such method is based on generic parameters (age, sex etc.) and does not allow for a “personalized” prediction of risk.

In addition, a number of novel plasma and urinary early biomarkers have recently been proposed for the post-operative diagnosis of AKI and its clinical outcomes in a variety of clinical settings.¹⁶⁻¹⁸

However, none of such markers has a pre-operative predictive value.

Therefore, there is the need to identify a reliable pre-operative biomarker of AKI. Preferably, the biomarker may be a parameter that can be corrected or normalized so that the risk of developing AKI is decreased.

To investigate whether EO and its regulatory gene network (RGN) are involved in the development of AKI and consequent death, the authors of the present invention investigated a large group of consecutive cardiac surgery patients. It is hypothesized that patients with elevated EO levels at baseline (i.e before the surgery) may have increased probability to develop AKI after an acute surgical stress because of an underlying asymptomatic renal damage.

The authors evaluated the predictive role of EO and related genetic polymorphisms of synthesis (Lanosterol Synthesis, LSS, 3β-hydroxyl-steroid dehydrogenase HSD), metabolism (P-glycoprotein encoded by MDR1) and activity (Na/Ca exchanger, SLC8A1, C-type transient receptor potential TRPC6, and adducins, ADDs) on renal dysfunction occurring post-operatively and all genetic polymorphisms have been selected before the surgery.

In addition, to verify if prolonged exposure to elevated EO plasma levels may affect kidney structure and function, the influence of chronic infusion of low doses of ouabain, in normal rats has been assessed.

### SUMMARY OF THE INVENTION

The present invention discloses that in patients carrying certain genetic polymorphisms, exposure to elevated levels of EO for a long period time correlates with the development of AKI after an acute hemodynamic stress such as cardiac surgery.

In order to investigate whether EO and RGN are involved in the development of AKI, its related death and other post-surgery complications, 425 cardiac surgery patients have been examined. All patients signed informed consent approved by HSR Ethical Committe. Blood samples for plasma EO and genotype determinations (GENEO profile) have been collected within 24 hours of the admission to the clinic. EO was extracted from plasma and measured by using a specific radioimmunoassay (MA). Furthermore, disease severity was scored according to European System for Cardiac Operative Risk Evaluation (EUROSCORE) and ACEF score (Age, Creatinine, and left ventricular Ejection Fraction). The results may be summarized as follows.

Concerning AKI following cardiac surgery:
425 patients underwent surgery with Cardiopulmonary By-pass (CBP): GENEO Profile was obtained in 374 patients.
   114/374 patients with GENEO profile (30.5%);
   260/374 patients without GENEO profile (69.5%);
95 patients developed AKI:
   49/114 patients with GENEO profile (43%);
   46/260 patients without GENEO profile (17%);
20/374 of the patients that developed AKI needed Renal Replacement Therapy (RRT) (5.3%).

Concerning in hospital mortality:
in hospital mortality was 2,4% (10 out of 425 patients died within 30 days);
70% (n.7) of dead patients were in the GENEO profile group;
in hospital mortality was 5.46 (CI 1.4-21.1 p=0.006) higher in patients carrying the GENEO profile than in those without, with a sensitivity of 70% and a specificity of 71%.

Therefore, the present invention relates to the identification of plasma Endogenous Ouabain (EO) levels and polymorphisms in related gene network (herein called GENEO) of EO synthesis (Lanosterol Synthesis, LSS rs914247, 3β-hydroxyl-steroid dehydrogenase HSD3B1 rs2236780), EO metabolism (P-glycoprotein encoded by MDR1 rs1045642) and EO activity (Na/Ca exchanger 1, SLC8A1 rs11893826, C-type transient receptor potential TRPC6 rs7925662, and adducins, ADD1 rs4961, ADD2 rs4984) as powerful predictive pre-operative biomarkers of Acute Kidney Injury (AKI) in patients undergoing cardiac and vascular surgery.

A subject is said to have the GENEO profile or to be GENEO or to be GENEO profile YES when carrying at least one of these combinations of SNPs:
ADD2 rs4984 [allelic genotype CT or TT] and HSD3B1 rs2236780 [allelic genotype GG] or;
ADD2 rs4984 [allelic genotype CT or TT] and SLC8A1 rs11893826 [allelic genotype AA] or;
LSS rs914247 [allelic genotype AA] and SLC8A1 rs11893826 [allelic genotype AA] or;
TRPC6 rs 7925662 [allelic genotype CC] and ADD1 rs4961 Trp [allelic genotype GT or TT] or;
plasma EO concentration >(greater than) 207 pM and MDR1 rs1045642 [allelic genotype TC or CC].

The GENEO profile, is a pre-operative biomarker that selectively identify those patients with a higher risk to develop post-operative AKI and who can benefit from targeted therapy. In addition, GENEO profile may provide information for a specific pre-operative therapy.

Compared to usual pre-operative stratification clinical score, GENEO have a greater sensitivity and specificity. Patients carrying the GENEO profile may benefit from specific pre-operative treatment.

In addition, the authors postulate that a pre-surgery therapy aimed at lowering EO levels in patients elected for cardiac surgery could improve the outcome of post-operative complications (specific pre-operative therapy with selective inhibitors of EO and its related gene network (GENEO) and drugs such as rostafuroxin).

It is therefore an object of the present invention a method for predicting the risk of developing acute kidney injury in a subject from which a biological sample is obtained comprising:
detecting the presence of at least one genomic single nucleotide polymorphism (SNP) selected from the group of: ADD1 rs4961 Trp [allelic genotype GT or TT], ADD2 rs4984 [allelic genotype CT or TT], HSD3B1 rs2236780 [allelic genotype GG], LSS rs914247 [allelic genotype AA], MDR1 rs1045642 [allelic genotype TC or CC], SLC8A1 rs11893826 [allelic genotype AA], TRPC6 rs7925662 [allelic genotype CC] from said biological sample,
   wherein the presence of said allelic genotype is predictive of the risk of developing acute kidney injury.

Preferably, the method further comprises measuring the level of endogenous ouabain in a biological sample, wherein a level greater than 207 pM is predictive of the risk of developing acute kidney injury.

Still preferably the presence of at least two genomic single nucleotide polymorphisms SNPs is detected.

In a preferred embodiment two genomic SNPs are detected and selected from:
ADD2 rs4984 [allelic genotype CT or TT] and HSD3B1 rs2236780 [allelic genotype GG]; or
ADD2 rs4984 [allelic genotype CT or TT] and SLC8A1 rs11893826 [allelic genotype AA]; or
LSS rs914247 [allelic genotype AA] and SLC8A1 rs11893826 [allelic genotype AA]; or
TRPC6 rs7925662 [allelic genotype CC] and ADD1 rs4961 Trp [allelic genotype GT or TT].

In a still preferred embodiment the genomic SNP detected is MDR1 rs1045642 [allelic genotype TC or CC].

Preferably the biological sample is a biological fluid or a tissue. The biological sample may be serum, urine or any other biological fluid.

Preferably the level of endogenous ouabain is measured by scintillation proximity assay. In a preferred embodiment the method of the invention predicts the risk of developing acute kidney injury after stress.

Preferably, the stress is an acute hemodynamic stress. Still preferably the stress is caused by a surgery. Yet preferably, the surgery is a cardiac or vascular surgery.

It is a further object of the invention a kit for predicting the risk of developing acute kidney injury according to the method of any one previous claims comprising reagents to detect at least one single nucleotide polymorphism (SNP) selected from the group of: ADD1 rs4961 Trp [allelic genotype GT or TT], ADD2 rs4984 [allelic genotype CT or TT], HSD3B1 rs2236780 [allelic genotype GG], LSS rs914247 [allelic genotype AA], MDR1 rs1045642 [allelic genotype TC or CC], SLC8A1 rs11893826 [allelic genotype AA], TRPC6 rs7925662 [allelic genotype CC].

Preferably, the kit comprises reagents to detect at least one combination of SNPs selected from the group of:
ADD2 rs4984 [allelic genotype CT or TT] and HSD3B1 rs2236780 [allelic genotype GG]; or
ADD2 rs4984 [allelic genotype CT or TT] and SLC8A1 rs11893826 [allelic genotype AA]; or
LSS rs914247 [allelic genotype AA] and SLC8A1 rs11893826 [allelic genotype AA]; or
TRPC6 rs7925662 [allelic genotype CC] and ADD1 rs4961 Trp [allelic genotype GT or TT]

Still preferably the kit further comprises means to measure endogenous ouabain level. Yet preferably the kit comprises reagents to detect the SNP MDR1 rs1045642 [allelic genotype TC or CC] and means to measure endogenous ouabain level.

It is a further object of the invention a selective ouabain inhibitor for use to decrease the risk of developing AKI in a subject at risk of developing AKI predicted according to the method of any one of claims 1 to 11.

Preferably the selective ouabain inhibitor is rostafuroxin or digibind.

It is a further object of the invention a method for decreasing the risk of developing AKI in a subject comprising:
predicting the risk of developing AKI according to the method of the invention;
if the subject is at risk, administering to said subject a selective endogenous ouabain inhibitor.

Preferably the selective endogenous ouabain inhibitor is rostafuroxin or digibind.

The SNPs detected in the present invention are all located in gene regions of the regulatory gene network of EO. The SNPs detected in the present invention all belong to a family of EO regulatory genes.

In the present invention, the selective endogenous ouabain inhibitor is rostafuroxin or digibind. However any selective endogenous ouabain inhibitor is suitable, such inhibitors are described in the references.¹⁹⁻²⁴

In the present invention a subject is said GENEO or GENEO profile YES when carrying at least one of these combinations of SNPs:
ADD2 rs4984 [allelic genotype CT or TT] and HSD3B1 rs2236780 [allelic genotype GG] or;
ADD2 rs4984 [allelic genotype CT or TT] and SLC8A1 rs11893826 [allelic genotype AA] or;
LSS rs914247 [allelic genotype AA] and SLC8A1 rs11893826 [allelic genotype AA] or;
TRPC6 rs 7925662 [allelic genotype CC] and ADD1 rs4961 Trp [allelic genotype GT or TT] or;
plasma EO concentration >(greater than) 207 pM and MDR1 rs1045642 [allelic genotype TC or CC]

The invention will be now illustrated by means of non-limiting examples referring to the following figures.

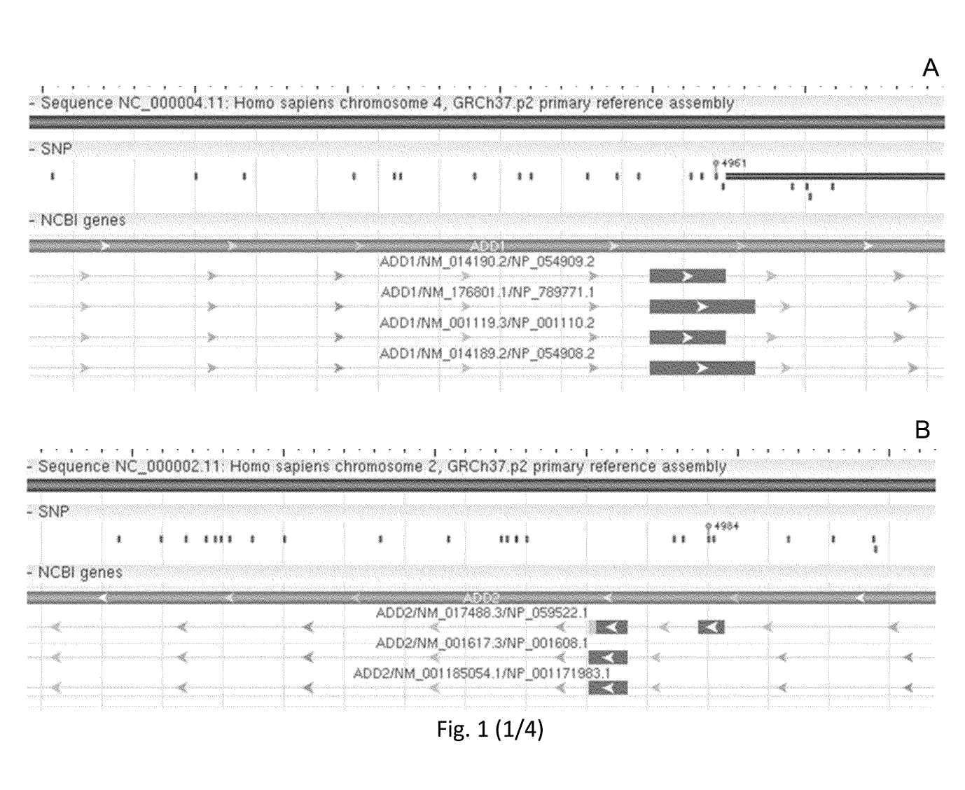

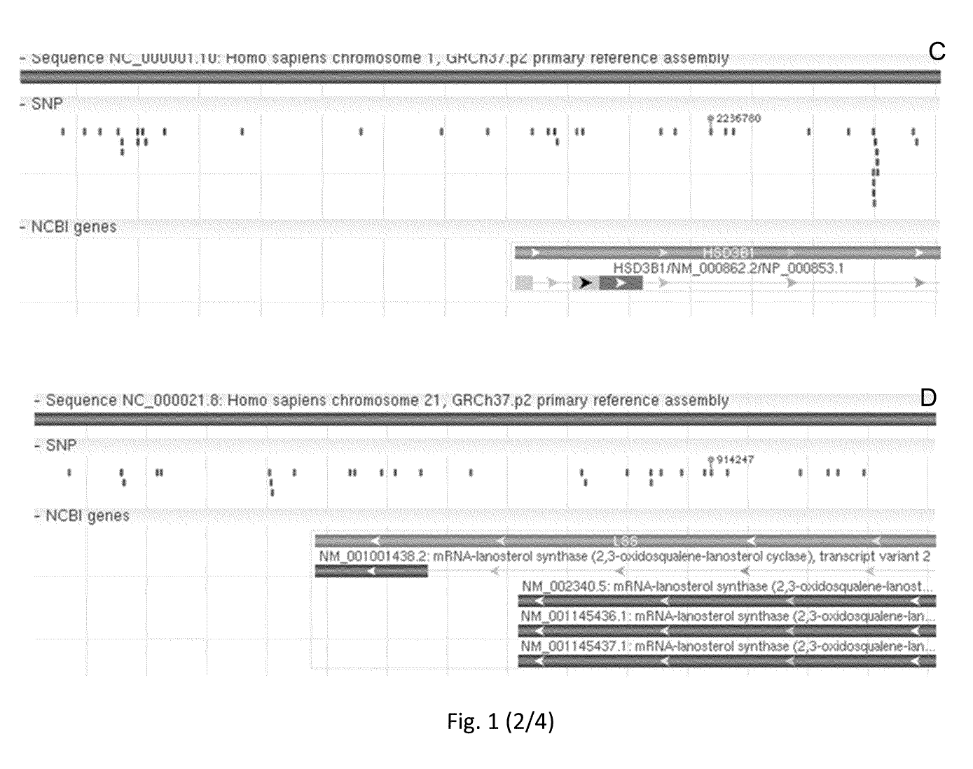

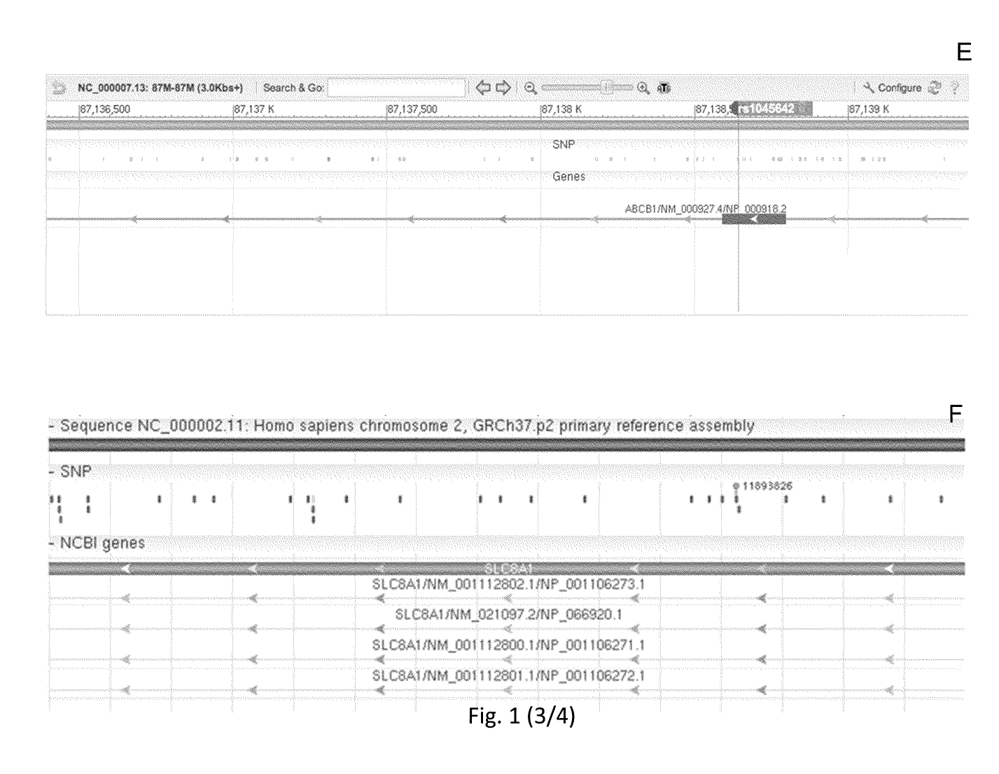

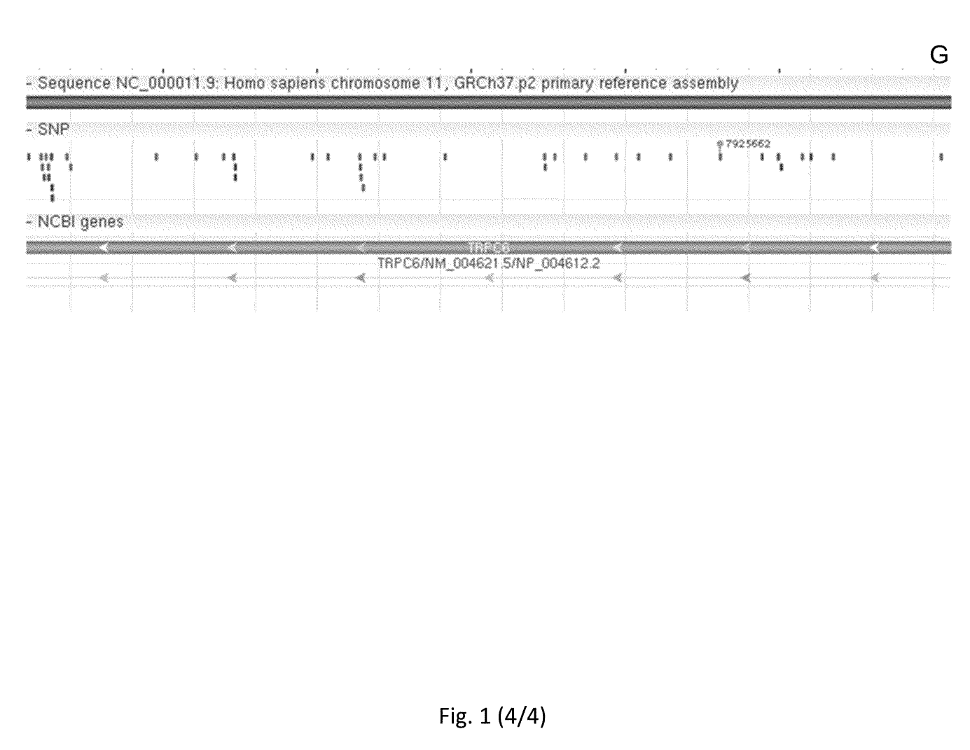

FIG. 1: Representation of SNP rs4961 (A), rs4984 (B), rs2236780 (C), rs914247 (D), rs1045642 (E), rs11893826 (F) and rs7925662 (G) on their respective gene and chromosome.

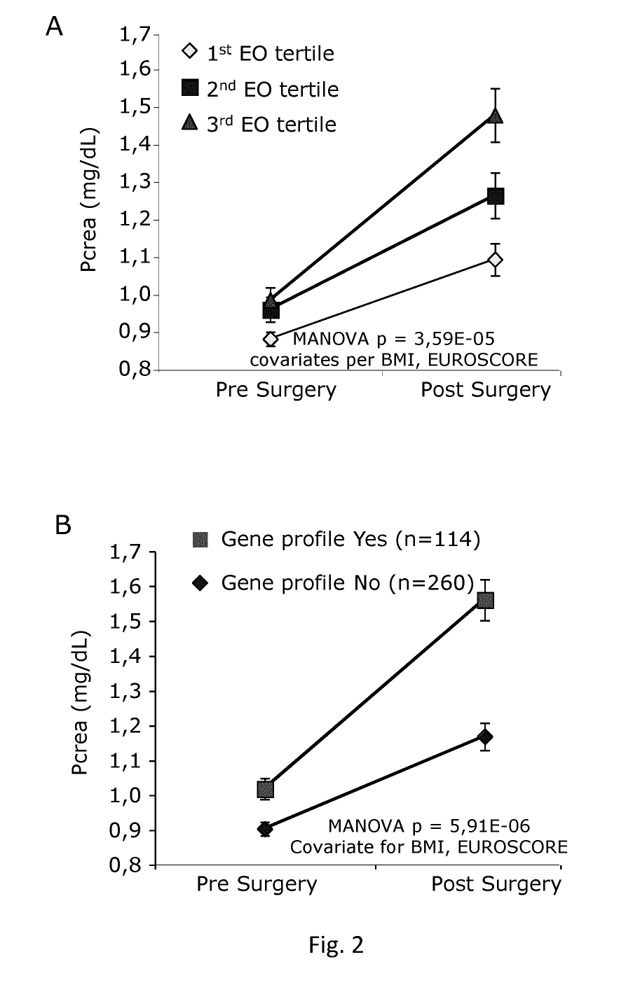

FIG. 2: Predictive effect of Endogenous Ouabain tertiles (A panel) and GENEO profile (B panel) on AKI development, measured by plasma creatinine concentration (mg/dL). Plasma EO tertile: all 425 patients were divided according to pre-operative plasma EO levels; 1st tertile <118 pmol/L, 2nd tertile 118-207 pmol/L, 3rd tertile >207 pmol/L; GENEO profile YES are patients carrying at least one of these combinations:
ADD2 rs4984 [allelic genotype CT or TT] and HSD3B1 rs2236780 [allelic genotype GG] or;
ADD2 rs4984 [allelic genotype CT or TT] and SLC8A1 rs11893826 [allelic genotype AA] or;
LSS rs914247 [allelic genotype AA] and SLC8A1 rs11893826 [allelic genotype AA] or;
TRPC6 rs 7925662 [allelic genotype CC] and ADD1 rs4961 Trp [allelic genotype GT or TT] or;
plasma EO concentration >(greater than) 207 pM and MDR1 rs1045642 [allelic genotype TC or CC].

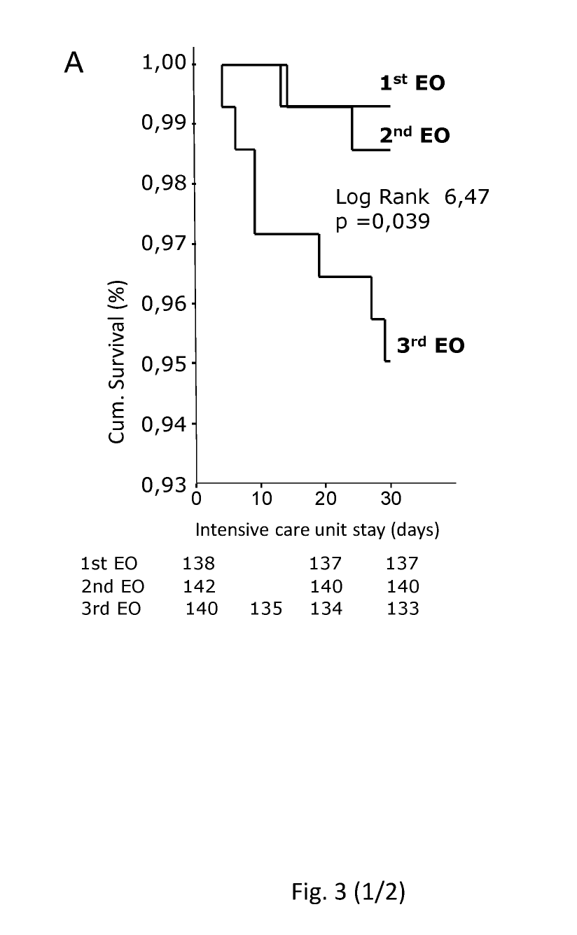

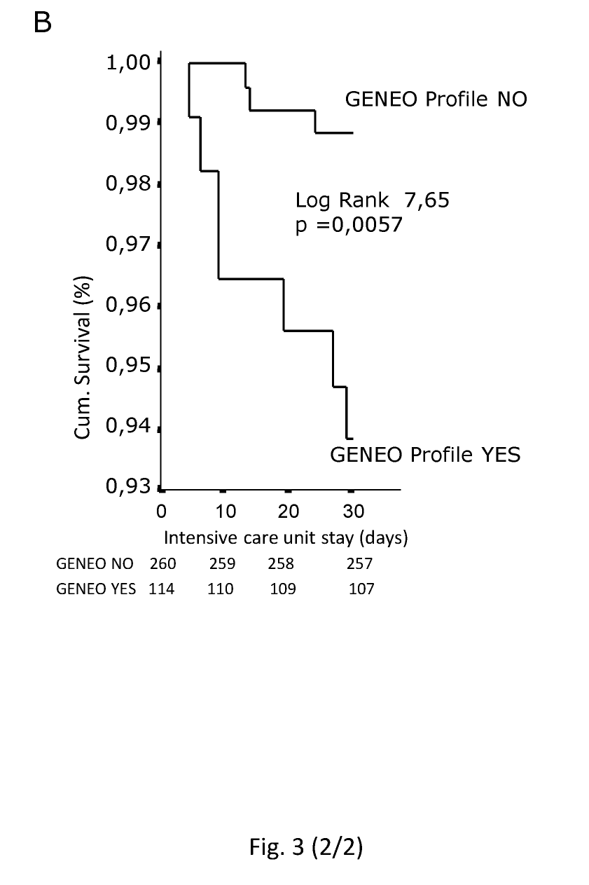

FIG. 3: Kaplan-Meier estimates of the cumulative probability of in-hospital mortality by EO tertile (panel A). EO tertile: all 425 patients were divided according to pre-operative plasma EO levels: 1st tertile <118 pmol/L, 2nd tertile 118-207 pmol/L, 3rd tertile >207 pmol/L. Patients with EO greater than 207 pmol/L had a 5% of in-hospital mortality i.e., had a greater risk of in-hospital mortality.

Kaplan-Meier estimates of the cumulative probability of in-hospital mortality by GENEO profile (panel B). GENEO profile includes 7 genes polymorphisms combinations ADD2 CT/TT & HSD3B1(a) GG or ADD2 CT/TT & SLC8A1 AA or LSS AA & SLC8A1 AA or TRPC6 CC & ADD1 Trp or EO>207 pM & MDR1 AG or GG. A greater risk to higher LHS (length of hospital stay) is present in these patients.

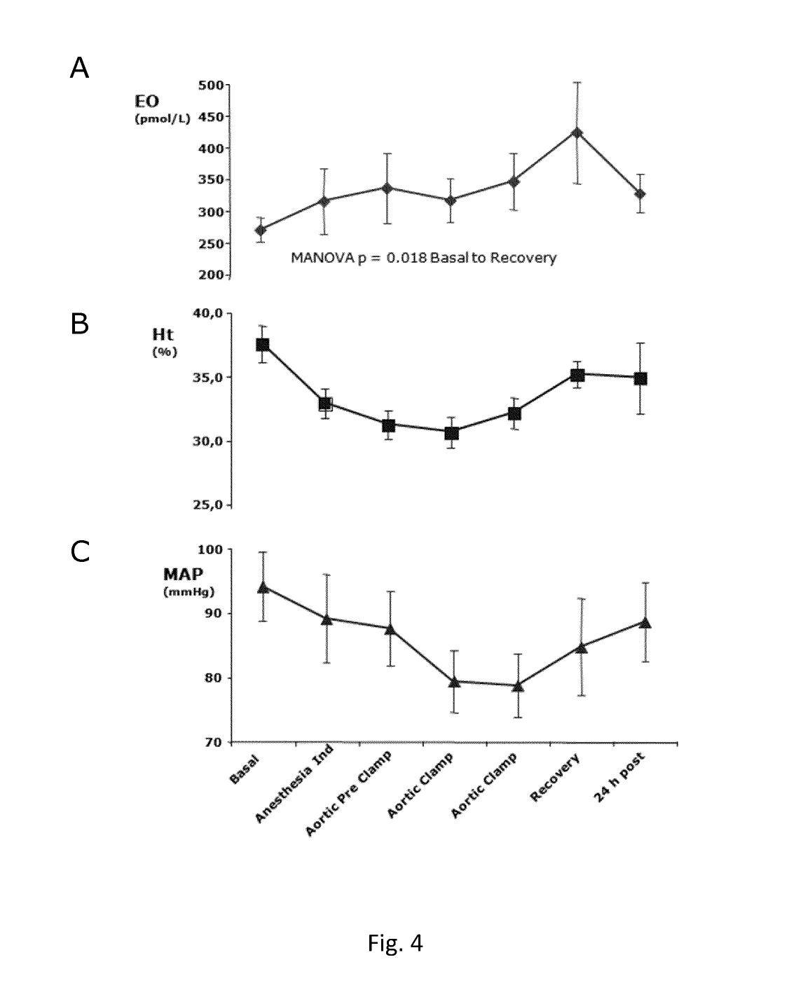

FIG. 4: (A) Plasma EO concentration, plasma EO raised after 15 min of anesthesia induction reaching the greater value after 4 hrs. Circulating EO tend to return at basal value 24 hours after aortic surgery. However, hematocrit (Ht, panel B) and mean arterial pressure (MAP, panel C), as expected, falls during aortic clamp.

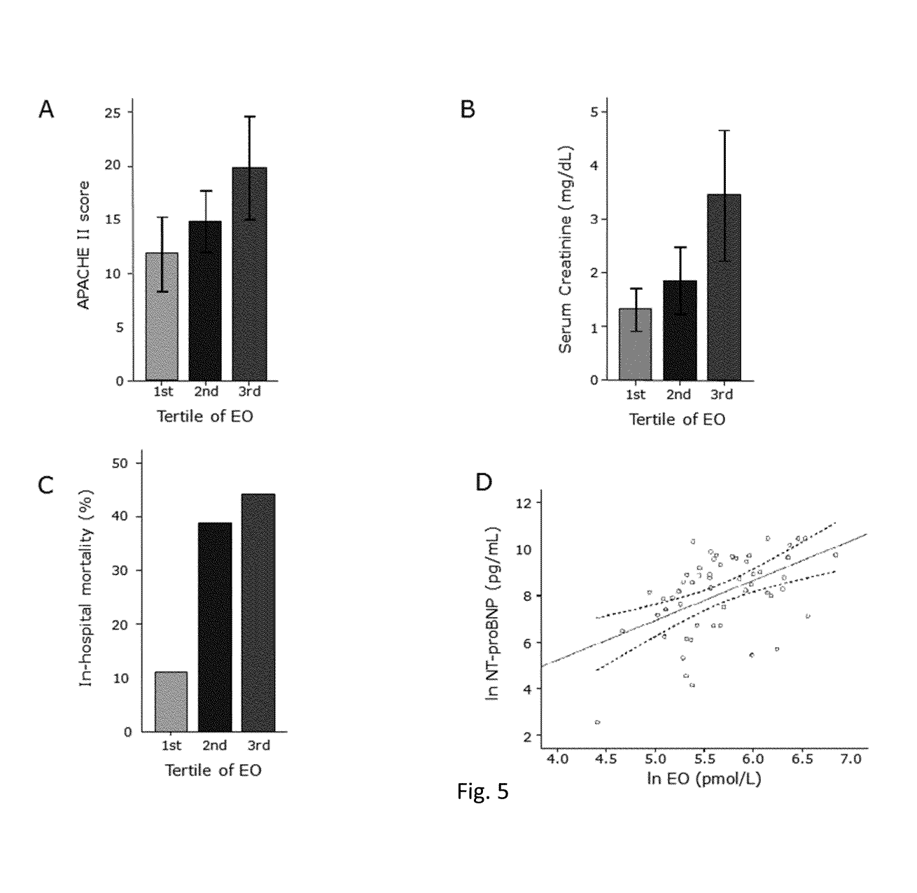

FIG. 5: Critical ill patients: APACHE II score (A panel, p=0.02) and in-hospital mortality (C panel, p=0.07) grouped by tertile of endogenous ouabain. Severity of illness correlated with EO. Serum creatinine (B panel, p=0.002) grouped by tertile of endogenous ouabain. Renal dysfunction is correlated with EO. Scatter plot (D panel) of NT-proBNP (log converted) vs. endogenous ouabain (log converted). EO is highly correlated with NT-proBNP.

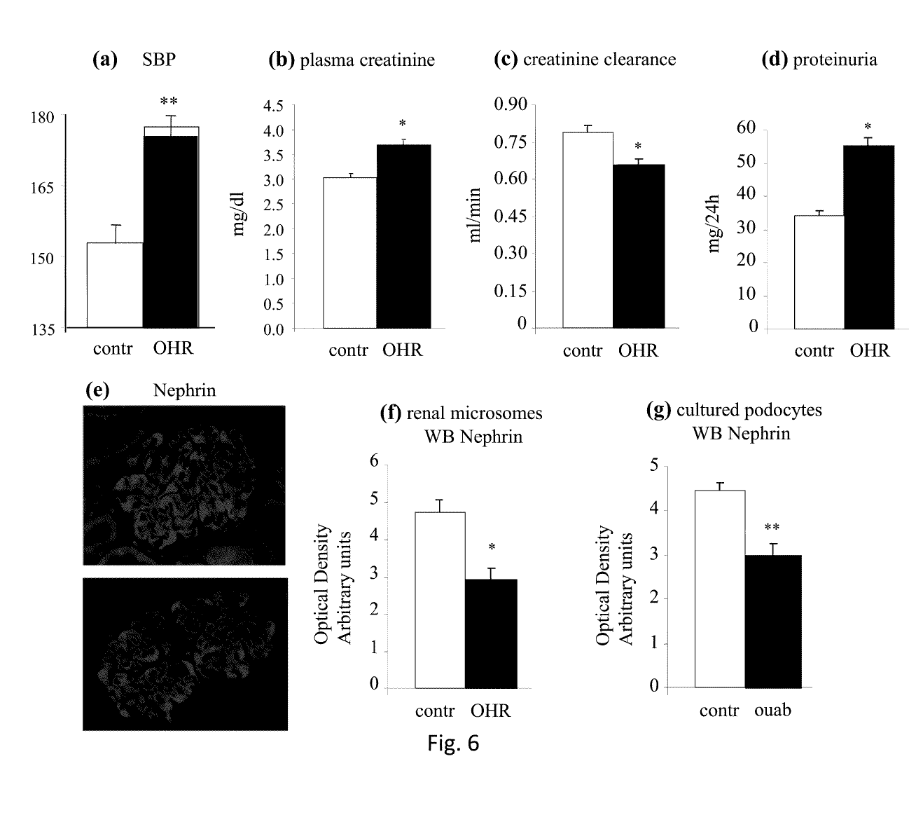

FIG. 6: Effects of ouabain on renal function and podocyte proteins in rats. Ouabain (15 μg/kg/day) was subcutaneously infused in normotensive Sprague-Dawley rats (OHR) for 8 weeks (n=7). Controls received saline (n=7). (a) Indirect systolic blood pressure (SBP), (b) plasma creatinine, (c) creatinine clearance and (d) urinary protein excretion were measured in controls and OHR rats. (e) Immunofluorescence analysis of the podocyte protein, nephrin, in kidney sections from controls and OHR rats. Three rats for each group were investigated. Magnification 1000×. (f) Western blot analysis of nephrin in renal microsomes from controls and OHR rats (10 μg protein/lane). Nephrin densitometric analysis, reported as arbitraty units, has been normalized for actin content. (g) Glomerular podocyte cultures, obtained from neonatal Sprague Dawley rats, were incubated with 10⁻⁹ M ouabain for 4 days. Nephrin densitometric analysis, reported as arbitraty units, has been normalized for actin content. Data are mean±sem of two separate experiments done in triplicate; *p<0.05, **p<0.01.

### DETAILED DESCRIPTION OF THE INVENTION

### Materials and Methods

### Human Study

### Study in Patients Undergoing Elective Cardiac Surgery

Four hundred twenty-five consecutive patients undergoing elective cardiac surgery were enrolled in the study after signed informed consent. This was a prospective observational study conducted at a single center from December 2007 to December 2009. The protocol was approved by the institutional San Raffaele Hospital Ethical Committee.

### Blood Samples

In addition to routine pre-operative assessments, blood samples were obtained for plasma EO and genotype determinations within 24 hours of admission to the clinic. Samples were stored at −80° C. until analysis. EO was extracted from plasma and measured by using a specific radioimmunoassay (MA) as previously described.²⁵

Scintillation Proximity Assay (SPA, Perkin Elmer) is hereby proposed in order to detect and quantify Plasma EO. SPA-EO method has been designated in order to convert existing radio-immuno assays (MA) to SPA. The SPA-EO method presents the advantages that no separation steps are needed and it does not use liquid of scintillation, contrary to existing methods. It consists in using Yttrium Silicate Beads conjugated with secondary antibody (Amersham anti-rabbit YSi SPA beads, cod.RPN140); these beads function as scintillators and are used as secondary antibody.

The secondary antibody on the beads binds the anti-ouabain antibody which then binds 3H-ouabain; the beads, due to their scintillation propriety, are activated and produce light so that they can be read on a beta-counter.

Briefly, the advantages of SPA towards MA are listed below:
minor duration of the experiment (20-24 h instead of about 72 h);
easier execution method implying minor variability;
important reduction of expensive and dangerous radioactive waste.

### Data Collection

An investigator blinded to the plasma biomarker concentration collected data from the patient chart notes, and the computerized data system. Disease severity was scored according to European System for Cardiac Operative Risk Evaluation (EUROSCORE)¹² and ACEF¹³ clinical scores (sex, age, creatinine, and left ventricular Ejection Fraction). EUROSCORE is a risk stratification system for the prediction of hospital mortality including age, sex, preoperative serum creatinine, extracardiac arteriopathy, chronic airway disease, severe neurological dysfunction, previous cardiac surgery, recent myocardial infarction, left ventricular ejection fraction, chronic congestive cardiac failure, pulmonary hypertension, active endocarditis, unstable angina.

ACEF score includes Age, pre-operative Creatinine and left ventricular Ejection Fraction for the prediction of mortality in elective cardiac operations. The ACEF score was computed as follows: age (years)/ejection fraction (%)+1 (if serum creatinine value was >2 mg/dL).

### Pre-Operative Evaluation and Intra-Operative Management

All patients underwent a pre-operative and intra-operative clinical evaluation as previously reported.²⁶

### Post-Operative ICU Management

After surgery, all patients were transferred to the intensive care unit, ICU. Plasma creatinine was measured every 6 hour until 48 hrs. AKI was defined according to the RIFLE criteria²⁷ and modified according to AKIN criteria²⁸ with new-onset of at least 1.5-fold increase or 0.3 mg increment of serum creatinine values from baseline. Plasma EO was measured in all patients that developed AKI, and in a control group without AKI.

Renal Replacement therapy (RRT)

In 15 patients undergoing continuous RTT, serial sampling for EO concentration, serum creatinine and the hematocrit were performed.

### Genotyping

Genomic DNA was extracted using standard methods. All subjects were genotyped for this set of SNPs relative to different genes.

The following table summarize the various SNPs analyzed and their relation to the GENEO genotype.

| SNP | | | |
|---|---|---|---|
| accession | | | |
| number | | Polynucleotide | GENEO |
| NCBI | | containing | allele |
| dbSNP | Gene (used abbreviation) | SNP | genotype |
| rs4961 | Adducin 1 (ADD1) | SEQ ID No. 1 | GT or TT |
| rs4984 | Adducin 2 (ADD2) | SEQ ID No. 2 | CT or TT |
| rs2236780 | hydroxy-delta-5-steroid | SEQ ID No. 3 | GG |
| | dehydrogenase, 3 beta- and | | |
| | steroid delta-isomerase 1 | | |
| | (HSD3B1) | | |
| rs914247 | lanosterol synthase (LSS) | SEQ ID No. 4 | AA |
| rs1045642 | MDR1 ATP-binding cassette, | SEQ ID No. 5 | TC or CC |
| | sub-family B (MDR/TAP), | | |
| | member 1 (MDR1) | | |
| rs11893826 | solute carrier family 8 | SEQ ID No. 6 | AA |
| | (sodium/calcium exchanger), | | |
| | member 1 (SLC8A1) | | |
| rs7925662 | transient receptor potential | SEQ ID No. 7 | CC |
| | cation channel, subfamily C, | | |
| | member 6 (TRPC6) | | |

Further details derived from NCBI dbSNP (http://www.ncbi.nlm.nih.gov/SNP) are reported below:

ADD1 rs4961 G/T: it is a missense polymorphism in Adducin 1 gene at position 2876505 on chr 4 as represented on FIG. 1A. The flanking sequence of the SNP is as follows, the SNP is indicated by the bold letter N:

(SEQ ID No. 1)
CAGCAGCGGG AGAAGACAAG ATGGCTGAAC TCTGGCCGGG
GCGACGAAGC TTCCGAGGAA N GGCAGAATGGAAGCAGTCCC
AAGTCGAAGA CTAAGGTGTG GACGAACATT ACACACGATC,
wherein N is G or T.

The allele frequency of minor allele in general population (HapMap CEU) is T 0.21 In the present invention, the GENEO profile allelic genotype is ADD1 Trp (GT or TT).

ADD2 rs4984 C/T: it is a synonymous polymorphism in Adducin 2 gene at position 70753911 on chr 2 as represented on FIG. 1B. The flanking sequence of the SNP is as follows, the SNP is indicated by the bold letter N:

(SEQ ID No. 2)
TTCAGAGACAGGACAGGAACGAGAGCCAGGCTCTGGTCCGGCCGTGTGCG
AGTTCTTCAG N GTTGCCCTCCACATCTGGAGTAACATATTGGTAGGTG
TGTTTTGATGAAGGAAACATGAC,
wherein N is C or T.

The allele frequency of minor allele in general population (HapMap CEU) is T 0.12 In the present invention, the GENEO profile allelic genotype is ADD2 CT or TT.

HSD3B1 rs2236780 A/G: it is an intronic polymorphism in hydroxy-delta-5-steroid dehydrogenase, 3 beta- and steroid delta-isomerase 1 gene at position 119851986 on chr 1 as represented on FIG. 1C. The flanking sequence of the SNP is as follows, the SNP is indicated by the bold letter N:

(SEQ ID No. 3)
ATGGCATAGTATGAAAGATACTGGGTGGGATTTCCAGAGACTAGATTCTG
GCCCTGACCC N GAACTTGAGAAGCAGCCACCTCAGCCTCCAGGCCTCT
TTTCTTCTCATCTAGAAAATCCC,
wherein N is A or G.

The allele frequency of minor allele in general population (HapMap CEU) is A 0.31 In the present invention, the GENEO profile allelic genotype is HSD3B1 GG

LSS rs914247 A/G: it is a 3′UTR polymorphism in lanosterol synthase gene at position 46434105 on chr 21, as represented on FIG. 1D. The flanking sequence of the SNP is as follows, the SNP is indicated by the bold letter N:

(SEQ ID No. 4)
AGGGGGAGAG GCCAGGGACT GCTACCTGCC CAGAAGGCGG
CAGGGAGGGG AAGAGCAGAT N AGGAGGTATAGGGTGTGCCCTGGGCA
AGGCAGCAGGGGTAACGAAGCTCT CAGTGACTCC,
wherein N is A or G.

The allele frequency of minor allele in general population (HapMap CEU) is A 0.32. In the present invention, the GENEO profile allelic genotype is LSS AA.

MDR1 rs1045642 C/T: it is a synonymous polymorphism in MDR1 ATP-binding cassette, sub-family B (MDR/TAP), member 1 gene at position 86976581 on chr 7 as represented on FIG. 1E. The flanking sequence of the SNP is as follows, the SNP is indicated by the bold letter N:

(SEQ ID No. 5)
CAGCATTGCTGAGAACATTGCCTATGGAGACAACAGCCGGGTGGT
GTCACAGGAAGAGAT N GTGAGGGCAGCAAAGGAGGCCAACATACAT
GCCTTCATCGAGTCACTGCCTAATGTAAGT,
wherein N is C or T,

The allele frequency of minor allele in general population (HapMap CEU) is C 0.46 In the present invention, the GENEO profile allelic genotype is MDR1 TC or CC.

SLC8A1 rs11893826 A/G: it is intronic polymorphism in solute carrier family 8 (sodium/calcium exchanger), member 1 gene at position 40418151 on chr 2, as represented on FIG. 1F. The flanking sequence of the SNP is as follows, the SNP is indicated by the bold letter N:

(SEQ ID No. 6)
AAGGAAGTCA GGTAAAAAAA GGAAAAGCAA GAAAAACAAC
AAACCACTCA CCTCCATAGC N CACTCAGTAA TGCAATGGTA
GTTAGCAGAT CCAATTACCA GCTACTCAGT GGCACTCAAT,
wherein N is A or G.

The allele frequency of minor allele in general population (HapMap CEU) is A 0.41. In the present invention, the GENEO profile allelic genotype is SLC8A1 AA.

TRPC6 rs7925662 C/T: intronic polymorphism in transient receptor potential cation channel, subfamily C, member 6 gene at position 100913516 on chr 11, as represented on FIG. 1G. The flanking sequence of the SNP is as follows, the SNP is indicated by the bold letter N:

(SEQ ID No. 7)
TAC CACATGGCCT TATGTGGATG TTTCTCTAGT CATTTTGGTC
ATCTGGGGCA CATTTTC N AGAAACTTCT TCATGAAGGG
ATCTTGGGAA CTATTTCCCT GAGTTCTTGT ATGTTGTTCA,
wherein N is C or T.

The allele frequency of minor allele in general population (HapMap CEU) is C 0.36. In the present invention, the GENEO profile allelic genotype is TRPC6 CC.

SNP genotypes were identified by the method of the 5′ nuclease allelic discrimination assays, TaqMan SNP Genotyping Assays using the ABI 7900HT Real-Time PCR System (Applied Biosystems, Foster City, Calif., USA).

Relative TaqMan® SNP Genotyping Assays, the indicated code is the Applied Biosystems assay ID:

| | |
|---|---|
| ADD1 rs4961 G/T | C_11764545_20 |
| ADD2 rs4984 C/T | C_2634640_10 |
| LSS rs914247 A/G | C_3270838_1_{—} |
| MDR1 rs1045642 C/T | C_7586657_20 |
| SLC8A1 rs11893826 A/G | C_2669606_10 |
| TRPC6 rs7925662 C/T | C_45029305_10 |

Relative primer sequence for custom assay:

HSD3B1 rs2236780 A/G:

(SEQ ID No. 8)
Forward GGTGGGATTTCCAGAGACTAGATTC
(SEQ ID No. 9)
Reverse GCTGAGGTGGCTGCTTCTC

### Statistical Analysis

Data are analyzed using SPSS 18.0 for Mac. The continuous data are expressed as mean±standard deviation. Dichotomous variable are presented as percentages. EO concentrations were log transformed to promote normality. Geometric mean and inter quartiles range (IQR) are presented. ANOVA was used to compare continuous variables among EO tertiles whereas chi-square analysis was used to compare discrete variables. Correlation and linear regression was applied to evaluate the relations among continuous variables. General linear model (Repeated Measures procedure MANOVA) assessed EO tertile and GENEO groups effect after correction for BMI and EUROSCORE on plasma creatinine values before and after surgery; results are displayed as means±SE.

Kaplan-Meier curves and Cox regression were used to compare hospital length of stay among the EO tertiles or GENEO profile groups. Log-rank statistic was used to test differences between groups and Hazard ratio was computed. Logistic regression analysis was used to examine the association of the EO tertiles and GENEO profile and to estimate relative risk with total AKI and hospital death. The models were adjusted for several baseline covariates, including BMI, and EUROSCORE. To measure the sensitivity and specificity for EO and GENEO, a conventional receiver-operating characteristic (ROC) curve was generated, and the area under the curve (AUC) was calculated.

### Study in Vascular Surgery Patients

As a validation population, critical ill patients from an independent and unrelated study were also selected. The aim of this study was to evaluate plasma endogenous ouabain in patients who underwent major elective vascular graft replacement of abdominal aorta. Nine patients underwent elective graft replacement of infra renal abdominal aortic aneurysm. Patients received a standardized anesthetic management. Monitoring included arterial and central venous blood pressure, ECG, temperature, pulse oximeter, end-tidal carbon dioxide, and urine output. General anesthesia was induced with fentanyl (2 mcg/kg) and propofol (2 mg/kg). To facilitate endotracheal intubation, rocuronium 0.5 mg/kg was administered. Anesthesia was maintained with sevofluorane or desfluorane (end-tidal concentration >1 MAC) and additional doses of fentanyl when required. Epidural anesthesia, when non controindicated, was performed preoperatively to ensure perioperative analgesia with a continuous perfusion of ropivacaine cloridrate 0.2% and sufentanyl 0.5 mcg/ml.

Further the authors measured EO concentrations in critically ill patients admitted to the medical intensive care unit (ICU) and studied their relationships to patient characteristics, end-organ function and natriuretic peptide.

This study was conducted at a single centre. Protocol was approved by the institutional review board of the University of Maryland. Adult patients admitted to the ICU were eligible if the patient did not have an acute cardiac condition (acute coronary syndrome, congestive heart failure, or arrhythmia). Patients transferred from other intensive care units were excluded. One hundred seventy-nine consecutive patients were enrolled in the study. Fifty-four patients had adequate blood samples for measurement of circulating ouabain. Data related to NT-proBNP has previously been published.⁽³⁶⁾

### Blood Samples

Blood samples were collected in additive free tubes to measure serum NT-proBNP and endogenous ouabain concentrations within 24 hours of admission to the ICU. The blood was immediately transported to the hospital's central lab where it was centrifuged and stored at −70° C. The NT-proBNP concentration was determined with an electrochemiluminescent immunoassay ElecSys 2010 (Roche Diagnostics, Mannheim, Germany). The interassay coefficient of variance (total imprecision) is <3.0%. The analytic range is from 20 to 35,000 pg/mL. Endogenous ouabain was extracted from plasma and measured by using a specific radioimmunoassay (MA) as previously described. The assay used an ouabain antiserum with low cross-reactivity for digoxin (approximately 0.42%) and spironolactone (<0.01%).

### Data Collection

An investigator blinded to the serum biomarker concentration collected data from the patient chart, nursing notes, and the computerized data system. The demographic, patient characteristics and clinical data included. Disease severity was scored according to the Acute Physiology and Chronic Health Evaluation (APACHE II) system, with higher values indicating more severe illness.(12) Echocardiography was obtained in thirty-three (61%) patients. A second investigator reviewed the chart after hospital discharge or death to ensure complete and accurate records.

### Statistical Analysis

The continuous data are expressed as mean±standard deviation. Dichotomous variable are presented as percentages. For the data analysis, NT-proBNP and EO concentrations were log transformed to promote normality. For the univariate analysis among tertiles, ANOVA (see p values in tables) was used to compare continuous variables and chi-square analysis was used to compare discrete variables. Multivariate stepwise regression analysis was performed to determine the independent relationship of variables with EO. Variables included in the analysis were NT-proBNP, serum creatinine, serum bilirubin, mean arterial pressure, heart rate, age and APACHE II score.

### Animal Study

Care and husbandry of rats complied with the European Directives No. 86/609 and with the Italian Law (DL116, Jan. 27, 1992). The authorization for animal use in Prassis Sigma-Tau laboratories was obtained from the Italian Health Authority. Ouabain hypertensive rats (OHR) were obtained by subcutaneous ouabain infusion (Sprague-Dawley rats, 15 μg/kg/day ouabain from Sigma-Aldrich, n=7) with osmotic mini-pumps for 8 weeks as described.^{1,2} Normotensive control rats received sterile saline (n=7). Systolic blood pressure and heart rate were recorded weekly in conscious rats by tail-cuff plethysmography.

### Biochemical Assays for Urinary Parameter Measurements

Rats were housed in single metabolic cages and 24 h-urine samples were collected and analyzed for total protein and creatinine excretion (kits from Sentinel Diagnostics, Milan, Italy) as described in Doi M, et al.²⁹ At sacrifice, rat plasma was collected for creatinine determination.

### Immunohistochemistry

Immunofluorescence was performed on rat kidney sections by using anti-nephrin and anti-synaptopodin antibodies as described.³⁰

### Renal Microsome Preparations

Renal microsomes were prepared from ouabain-infused rats and saline controls as described³⁰ and analyzed by Western blotting.

### Podocyte Isolation

Kidneys were taken from 7-10 day-old rats and glomerular podocytes were isolated and cultured as described.³⁰ Cell characterization was performed using markers of podocytes (nephrin, podocin, synaptopodin), epithelial (cytokeratin), smooth muscle (a-SMC) and endothelial cells (CD31). Podocytes were incubated in the absence or presence of 10⁻⁹ M ouabain for 4 days and analyzed by Western blotting.

### Western Blotting

Samples were analyzed by Western blotting using specific antibodies against podocyte markers, such as nephrin (Santa Cruz Biotechnology), podocin (Sigma-Aldrich), synaptopodin (Progen Biotechnik), ZO-1 (Invitrogen) as described in Ferrandi M, et al (J Mol Med 2010; 88:203-217).³⁰ Actin (Sigma-Aldrich) was used for normalization.

### Results

Pre-operative and post-operative patient characteristics are presented in Table 1. Cardio pulmonary by-pass was used in 86.3% of 425 cardio-surgery patients. Custodiol cardioplegia was used for 76% of patients and in the remaining cases, Buckberg cardioplegia was used.

TABLE 1
Clinical characteristics of the study participants

| | Pre Operatve | | | |
|---|---|---|---|---|
| SEX (f/m) | 161/264 | | | |
| AGE (yrs) | 62.1 ± 13.8 | | | |
| BMI (kg/m²) | 25.0 ± 4.04 | | | |
| FE (%) | 57.0 ± 10.5 | | | |
| PCreatinine (mg/dL) | 0.93 ± 0.34 | | | |
| eGFR (ml/m/1.73) | 73.7 ± 22.4 | | | |
| EUROSCORE | 4.90 ± 5.17 | | | |
| ACEF | 1.17 ± 0.45 | | | |
| EO (pmol/L) | 160 | (68-456) | | |
| Surgical planning | | | | |
| Mitral valve repair | 131 | (30.8%) | | |
| CABG: | 60 | (14.1%) | | |
| Combined surgery | 84 | (18.8%) | | |
| AAO | 42 | (9.8%) | | |
| Others | 16 | (3.7%) | | |
| Previous cardiac surgery | 53 | (12.5%) | | |
| Prior Medical History | | | | |
| Hypertension | 196 | (46.1%) | | |
| Chronic kidney disease | 27 | (6.3%) | | |
| Diabetes | 46 | (10.8%) | | |
| NYHA I. II. III. IV (%) | 24 | 51 | 23 | 2.4 |

| Intra Operative | |
|---|---|
| Surgery time (min) | 263.9 ± 79.0 |

| | | |
|---|---|---|
| CPB (%) | 390 | (86.3%) |
| Custodiol cardioplegia | 323 | (76.1%) |

| | |
|---|---|
| CPB time (min) | 93.2 ± 35.5 |
| ACCT (min) | 70.3 ± 25.8 |
| MAP (mmHg) | 71.8 ± 6.4 |

| | | |
|---|---|---|
| Post Operative | | |
| Inotrops (n^{o}) | 180 | (42.4%) |
| Diuretics (n^{o}) | 75 | (16.6%) |
| IABP (n^{o}) | 18 | (3.9%) |
| Transfusion (n^{o}) | 66 | (15.5%) |

| | |
|---|---|
| EO (pmol/L) | 289 |
| | (153-626) |
| P Creatinine (mg/dl) | 1.27 ± 0.73 |
| Troponine T peak | 2.22 ± 3.34 |
| ICU stay (days) | 3.22 ± 5.61 |
| LHS (days) | 11.9 ± 11.8 |

| | | |
|---|---|---|
| AKI (%) | 103 | (24.2%) |
| RRT (%) | 20 | (4.7%) |
| Mortality (%) | 10 | (2.35%) |

Combined surgery is valve repair/replacement joined with coronary-artery bypass or multiple valve replacements/repairs.
Closure of inter-atrial septal defects, excision of intra-myocardial masses, aneurysm-ectomy and atrial fibrillation radiofrequency ablation are included in the category “other”.
AAR: ascending aorta replacement.
ACCT: Aortic cross-clamp time.
AKI: acute kidney injury,
CABC: Coronary Artery Bypass.
GraftCKD: chronic kidney disease.
CPB: cardiopolmonary by pass.
CPB: cardiopulmonary by-pass.
IABP: inotropic arterial blood pressure.
ICU: intensive care unit stay.
LHS: Length of Hospital Stay.
MAP: mean arterial pressure.
NYHA: New York heart association functional classification.
RRT: replacement renal therapy.

AKI incidence, according to the criterion R of RIFLE²⁷ was 24%. 4.7% of the patients that developed AKI needed RRT. All of the patients who died developed AKI.

Patients in the highest EO tertile (plasma EO>207 pmol/L) had a relative risk of AKI that was 5.1 (CI % 2.64-9.84, p=1.16E-6) fold higher than in those in the lowest tertile.

In cardiac and vascular surgery patients, plasma EO showed a progressive increase until 10 hours after surgery, starting 15 minutes after CPB. The EO increase was similar in the two surgery conditions, despite the marked difference in volume expansion, expressed as hematocrit variation. However, in 11 patients in whom they were measured, proBNP and catecholamines started rising after 24 hrs, when EO starts to decrease after its early increase.

Pre-operative EO values are strictly related (r=0.484) to the EO post-operative values. Plasma EO increased both in patients without AKI (246.7 pmol/L IQR 142-446) and in patients who developed AKI (335.9 pmol/L, IQR 184-703, p<0.001). Post-operative plasma creatinine (which is considered a prognostic value of AKI in the present study) related to baseline EO concentrations (β=0.217 p=6.28E-06) and increased with each tertile of baseline EO (1.09±0.5 vs. 1.24±0.7 vs. 1.47±0.84, p=4.36E-05, Table 4). Patients had increasing prevalence of acute renal failure, renal replacement therapy (RRT), in-hospital mortality, ICU stay and length of hospital stay with each incremental tertile of EO, as shown in Table 2.

TABLE 2
Clinical characteristics of 425 CABG (coronary artery bypass graft) patients
divided according to tertile preoperative EO values (1st tertile <118 pmol/L, 2nd tertile
118-207 pmol/L, 3rd tertile >207 pmol/L)

| | | |
|---|---|---|
| 1^{st} EO Tertile | 2^{nd} EO Tertile | 3^{rd} EO Tertile |

Pre Operative

| Sex (f/m) | 58/86 | (13.6%) | 44/96 | (10.4%) | 59/82 | (13.9%) |
|---|---|---|---|---|---|---|
| Age (yrs) | 61.4 ± 13.7 | | 62.6 ± 13.8 | | 62.2 ± 13.7 | |
| BMI (Kg/m²) | 25.3 ± 3.51 | | 24.8 ± 3.84 | | 25.0 ± 4.51 | |
| EF % | 58.2 ± 9.4 | | 55.8 ± 11.5 | | 56.7 ± 10.4 | |
| EUROSCORE | 4.56 ± 4.18 | | 4.64 ± 4.46 | | 5.52 ± 6.51 | |
| ACEF | 1.10 ± 0.38 | | 1.22 ± 0.53 | | 1.17 ± 0.42 | |
| PCrea | 0.87 ± 0.22 | | 0.95 ± 0.40 | | 0.98 ± 0.38 | |
| eGFR (ml/m 1.73) | 76.4 ± 1.6 | | 73.4 ± 1.9 | | 71.2 ± 2.0 | |
| P EO (pmo/L) | 82.7 | (40-118) | 160.1 | (130-198) | 313.8 | (222-534) |

| | | | |
|---|---|---|---|
| Hypertension (%) | 13.4 | 17.9 | 14.8 |
| CKD (%) | 0.9 | 2.8 | 2.6 |
| Diabetes (%) | 1.9 | 4.9 | 4.0 |
| NYHA I. II. III. IV (%) | 28/46/24/2 | 22/52/24/1 | 21/55/21/4 |

Operative

| | | | |
|---|---|---|---|
| Surgery time (min) | 232.0 ± 60.3 | 262.7 ± 88.5 | 276.9 ± 77.4 |
| CBP time (min) | 92.7 ± 35.1 | 92.7 ± 35.3 | 93.1 ± 35.3 |
| ACC time (min) | 68.3 ± 25.5 | 70.6 ± 27.8 | 72.6 ± 23.6 |
| MAP (mmHg) | 70.8 ± 5.8 | 72.2 ± 6.4 | 72.5 ± 6.9 |
| Inotrops (%) | 14.1 | 11.9 | 16.9 |
| Diuretics (%) | 1.7 | 2.1 | 2.6 |
| IABP (%) | 1 | 1.4 | 1.9 |
| Transfusion (%) | 5 | 5.5 | 5 |

Post-Operative

| P Creatinine (mg/dl) | 1.09 ± 0.50 | | 1.24 ± 0.73 | | 1.47 ± 0.84* | |
|---|---|---|---|---|---|---|
| P EO (pmo/L) | 213.9 | (109-445) | 264.4 | (154-437) | 364.8 | (206-698)* |
| Troponine T peak | 2.34 ± 0.37 | | 2.04 ± 0.23 | | 2.30 ± 0.22 | |
| ICU stay (days) | 1.0 | (NC) | 1.0 | (NC) | 2.0 | (1.42-2.58)^{#} |
| LHS (days) | 8.0 | (7.42-8.58) | 8.0 | (7.15-) | 10.0 | (8.82-11.18)^{#} |

| | | | |
|---|---|---|---|
| AKI (%) | 4 | 7.3 | 12.9* |
| RRT (%) | 0.7 | 1.2 | 2.8* |
| IHM (%) | 0.2 | 0.5 | 1.6* |

Patient pre operative characteristics by EO tertile are shown in the top section, operative in the middle, and post operative in the bottom section.
IHM: in hospital mortality.
Values are mean ± SEM.
Plasma EO is geometric mean (Inter Quartile Range)
*Post Hoc Test Bonferroni <0.05 vs 1st and 2nd EO Tertile
^{#}Log-Rank Test. median (CI 95%). p < 0.01
RRT = replacement renal therapy
BMI = body mass index
EF = ejection fraction
EUROSCORE = European System for Cardiac Operative Risk Evaluation
ACEF = Age, Creatinine and left ventricular Ejection Fraction
eGFR = extimated Glomerular Filtration Rate
P EO = Plasma EO
CKD = cronic kidney disfuction
ICU = Intensive care unit stay
IABP = inotropic arterial blood pressure
NYHA = New York heart association
CPB: cardiopulmonary by-pass
ACC = Aortic cross-clamp
MAP = mean arterial pressure
LHS = Length of Hospital Stay

Predictive effect of EO on plasma creatinine is reported in FIG. 1, A panel. Those patients in the highest tertile (plasma EO>207 pmol/L) display a greater increase in plasma creatinine (MANOVA within=0.032, and between=0.026 subjects, including covariates for BMI, EUROSCORE). Multiple logistic regression analysis showed that increased baseline EO tertiles were associated with AKI (Table 2). Associations remained independently significant after adjustments for significant covariates (Table 3).

TABLE 3
Logistic Regression
Variables in the Equation

| | | B | S.E. | Wald | df | Sig. | Exp(B) |
|---|---|---|---|---|---|---|---|
| Step | TERTEO | | | 24.572 | 2 | .000 | |
| 1 | TERTEO(1) | .760 | .370 | 4.221 | 1 | .040 | 2.137 |
| | TERTEO(2) | 1.689 | .353 | 22.910 | 1 | 1.67e−006 | 5.413 |
| | AGE | .011 | .013 | .671 | 1 | .413 | 1.011 |
| | TOS | .455 | .100 | 20.574 | 1 | .000 | 1.576 |
| | NYHA | .501 | .189 | 7.004 | 1 | .008 | 1.650 |
| | HTP | .643 | .288 | 4.966 | 1 | .026 | 1.902 |
| | CKD | −.082 | .511 | .026 | 1 | .872 | .921 |
| | DM | −.209 | .403 | .268 | 1 | .605 | .812 |
| | STROKE | .789 | .560 | 1.986 | 1 | .159 | 2.201 |
| | eGFR_B | −.029 | .008 | 13.767 | 1 | .000 | .971 |
| | Constant | −2.717 | 1.237 | 4.824 | 1 | .028 | .066 |

Variables in the Equation

| | | B | S.E. | Wald | df | Sig. | Exp(B) |
|---|---|---|---|---|---|---|---|
| Step | GENEO | 1.239 | .293 | 17.865 | 1 | 2.37e−005 | 3.452 |
| 1 | AGE | .008 | .014 | .348 | 1 | .555 | 1.008 |
| | TOS | .489 | .109 | 20.234 | 1 | .000 | 1.631 |
| | NYHA | .497 | .197 | 6.377 | 1 | .012 | 1.644 |
| | HPT | .710 | .303 | 5.481 | 1 | .019 | 2.035 |
| | CKD | .132 | .552 | .057 | 1 | .811 | 1.141 |
| | DM | −.042 | .420 | .010 | 1 | .920 | .959 |
| | STROKE | .047 | .603 | .006 | 1 | .937 | 1.048 |
| | EGFR_B | −.028 | .008 | 11.164 | 1 | .001 | .972 |
| | Constant | −3.024 | 1.331 | 5.160 | 1 | .023 | .049 |

a Variable(s) entered on step 1: ETA, TIPOINTE, NYHA, IPT, IRC, DM, STROKE, EGFR_B.
HPT = hypertension
CKD = cronic kidney disfuction
TOS = type of surgery
eGFR_B = extimated Glomerular Filtration Rate Baseline (before surgery)
DM = diabetes mellitus

Within 30 days, death occurred in 7 (4.96%) of 141 patients in the 3^{rd} EO tertile group, 1 (0.69%) of 140 patients in the 1^{st}, and 2 (1.43%) of 144 patients in 2^{nd} EO tertile group. The relative risk in the highest EO group was 6.15 (as compared to the lowest tertile); 95% confidence interval [CI], 0.70 to 53.8; p=0.05 (FIG. 2, A panel).

Pre-operative characteristics of patients by allele combinations GENEO profile (114 patients, 30.5% of 374) of 7 genes:
ADD2 [allelic genotype CT or TT] and HSD3B 1 [allelic genotype GG] or;
ADD2 [allelic genotype CT or TT] and SLC8A1 [allelic genotype AA] or;
LSS [allelic genotype AA] and SLC8A1 [allelic genotype AA] or;
TRPC6 [allelic genotype CC] and ADD1 Trp [allelic genotype GT or TT] or;
plasma EO concentration >207 pM and MDR1 [allelic genotype TC or CC] are reported in Table 4.

TABLE 4
GENEO clinical characteristics

| | GENEO YES | | | |
|---|---|---|---|---|
| | (n = 114) | | GENEO NO (n = 260) | |
| Sex (f/m) | 43/71 | | 102/158 | |
| Age (yrs) | 63.8 ± 13.5 | | 61.9 ± 13.8 | |
| BMI (Kg/m²) | 24.8 ± 4.28 | | 24.9 ± 3.68 | |
| FE % | 56.6 ± 10.86 | | 57.1 ± 10.51 | |
| EUROscore | 6.28 ± 7.19* | | 4.55 ± 4.19 | |
| ACEF | 1.22 ± 0.45 | | 1.15 ± 0.45 | |
| PCrea pre surgery | 1.02 ± 0.44* | | 0.89 ± 0.27 | |
| eGFR (ml/m/1.73) | 67.7 ± 2.18* | | 76.1 ± 1.33 | |
| EO (pmol/L) | 252.9 | (115-523) | 134.2 | (67-249) |
| Hypertension (%) | 46.5 | | 46.5 | |
| CKD (%) | 10.5* | | 5 | |
| Diabetes (%) | 13.2 | | 10.4 | |
| NYHA I. II. III. IV (%) | 20/53/23/4 | | 26/51/21/2 | |
| Surgery lenght (min) | 280 ± 15.3 | | 242 ± 16.3 | |
| CBP time (min) | 101.1 ± 4.19* | | 89.7 ± 2.67 | |
| ACC time (min) | 74.9 ± 2.89 | | 68.1 ± 1.93 | |
| MAP (mmHg) | 73.4 ± 0.83* | | 71.4 ± 0.46 | |
| Inotrops (%) | 16.3 | | 26.1 | |
| Diuretics (%) | 3 | | 3.8 | |
| IABP (%) | 1.9 | | 3 | |
| Transfusion (%) | 8 | | 7.5 | |
| EO (pmol/L) | 360* | (199-495) | 253 | (142-568) |
| P Creatinine (mg/dl) | 1.58 ± 0.08* | | 1.15 ± 0.04 | |
| Troponine | 2.10 ± 0.25 | | 2.47 ± 0.20 | |
| ICU stay (days) | 2.0 | (1.31-2.69)^{#} | 1 | (NC) |
| LHS (days) | 10.0 | (8.64-11.36)^{#} | 8.0 | (7.41-8.59) |

| | | |
|---|---|---|
| AKI (%) | 43.1* | 17.7 |
| RRT (%) | 12.3* | 2.3 |
| Mortality (%) | 6.1* | 1.2 |

Values are mean ± SEM.
EO = geometric mean (IQR).
*p < 0.05 ANOVA
^{#}Log-Rank Test. median (CI 95%).
p < 0.001

Anthropometric parameters were similar, while EUROSCORE and renal function correlated significantly with the GENEO group. FIG. 1, B panel presents the creatinine plasma levels in the two groups before and after surgery, showing a strong effect of GENEO profile. Among patients who develop post-operative AKI, 49 (51.6% p=4.26E-07) were patients carrying the GENEO profile.

Kaplan-Maier survival curve of in hospital mortality was 5.46 (CI 1.4-21.1p=0.006) higher, with a sensitivity of 70% and a specificity of 71% in those patients carrying the GENEO profile (FIG. 2, B panel). Logistic regression analysis showed that EO tertile was the strongest predictor of in hospital mortality after correction for covariates (Table 5). Conventional ROC curves for AKI and for in hospital mortality were generated for plasma pre-operative EO and for GENEO profile. The AUCs of the two ROC curves are 0.70 (p=1.02E-07) and 0.72 (p=1.02E-07), respectively.

TABLE 5
Risk of AKI and Mortality Logistic Linear Regression

| | AKI | | In Hospital Motality | |
|---|---|---|---|---|
| Event Category | Relative Risk (95% CI) | | Relative Risk (95% CI) | |
| parameters | Unadjusted (95% CI) | Adjusted (95% CI) | Unadjusted (95% CI) | Adjusted (95% CI) |
| 3^{rd} EO tertile | 4.78 (2.60-8.78) | 5.02 (2.63-9.85) | 7.46 (0.91-61.3) | 7.13 (0.77-65.8) |
| GENEO | 3.51 (2.15-5.71) | 3.39 (2.00-5.73) | 5.60 (1.42-22.1) | 6.56 (1.24-34.7) |
| EUROSCORE | 1.13 (1.08-1.19) | 1.09 (1.03-1.15) | 1.14 (1.06-1.21) | 1.09 (1.00-1.18) |
| ACEF | 4.67 (2.69-8.12) | 4.27 (2.35-7.75) | 2.77 (1.10-6.96) | 2.90 (0.94-8.97) |
| CBP | 0.63 (0.28-1.39) | 0.88 (0.35-2.29) | 0.68 (0.08-5.56) | 1.07 (1.11-10.1) |

Relative risk of acute kidney injury (AKI) and in hospital mortality over the entire observational study population of 425 consecutive cardiac surgery patients. Unadjusted relative risk were estimated with the use of logistic regression. Adjusted relative risk were estimated with the use of logistic regression. EO tertile and GENEO with adjustment for body mass index

EUROSCORE is a risk stratification system for the prediction of hospital mortality including age, sex, preoperative serum creatinine, extracardiac arteriopathy, chronic airway disease, severe neurological dysfunction, previous cardiac surgery, recent myocardial infarction, left ventricular ejection fraction, chronic congestive cardiac failure, pulmonary hypertension, active endocarditis, unstable angina.

ACEF score was computed as follows: age (years)/ejection fraction (%)+1 (if serum creatinine value was >2 mg/dL). EUROSCRORE was adjusted for BMI. EO tertile and GENEO. ACEF was adjusted for sex and BMI. EO tertile and GENEO.

Plasma endogenous ouabain was also evaluated in patients underwent major elective vascular graft replacement of abdominal aorta. It was observed that plasma EO starts to rise after anesthesia induction reaching the main increase during the recovery (from 270.4±20 to 423.6±80 pmol/L, p repeated measure=0.018) as show in FIG. 4A. Circulating EO tend to return at basal value 24 hours after aortic surgery. However, hematocrit (Ht, FIG. 4B) and mean arterial pressure (MAP, FIG. 4C), as expected, falls during aortic clamp. Indeed, intraoperative management objectives are to preserve organs perfusion and to maintain an adeguate intravascular volume and cardiac output. It is also important to anticipate the surgical maneuvers, like aortic cross-clamp and unclamping, that alter blood pressure and intravascular volume. For these reasons intraoperatively are used vasodilators (nitroglycerine, nitroprusside), crystalloids (Ringer acetate and physiological solution) and when needed blood transfusions, depending on the different stages of surgery. In our patients total blood loss were 910±512 ml, total infusion during surgery 3731±987 ml and total urinary output 729±344 ml. Plasma creatinine values did not change. Surgery lasted 150±72 minutes and aortic cross clamp time was 40±17 minutes. Following all these intra operative maneuvers MAP and volume expansion are expected to have acute modification.

In conclusion, these findings support that EO intraoperative increase is related to acute hemodinanic modifications.⁽³⁵⁾ In particular, volume expansion and blood pressure fall are the two main stimuli for EO adrenal secretion.

Further the authors measured EO concentrations in critically ill patients admitted to the medical intensive care unit (ICU) and studied their relationships to patient characteristics, end-organ function and natriuretic peptide.

The patient characteristics are presented in Table 6.

TABLE 6
Critically ill Patient Characteristics

| | |
|---|---|
| Age (yr) | 57 ± 16 |
| Male | 57% |
| African American | 56% |
| Mean arterial pressure (mmHg) | 80 ± 21 |
| Heart rate (bpm) | 97 ± 25 |
| EO (pmol/L)) | 270 (204-411) |
| NT-proBNP (pg/mL)) | 4565 (1151-13309) |
| Creatinine (mg/dL) | 2.2 ± 1.9 |
| Total bilirubin (mg/dL) | 2.1 ± 3.7 |
| APACHE score | 15.5 ± 8.6 |
| Ejection fraction (%) | 52 ± 17 |
| Left ventricular hypertrophy | 24% |
| In-hospital death | 32% |
| Acute medical diagnosis | |
| Acute Kidney Injury | 37% |
| Acute liver failure | 17% |
| Gastrointestinal bleeding | 19% |
| Pneumonia | 44% |
| Acute respiratory distress syndrome | 37% |
| Sepsis | 43% |
| Shock | 19% |
| ICU interventions | |
| Mechanical ventilation | 63% |
| Intravenous vasopressors | 22% |
| Transfusion of blood products | 24% |
| Volume resuscitation | 61% |
| Antibiotics | 69% |
| Steroids | 30% |

The patients were diverse, with 57% male and 56% African-American. The mean age was 57±16 years. The most common admission diagnoses to the ICU were sepsis (43%), pneumonia (44%), and acute respiratory distress syndrome (37%). Most patients required mechanical ventilation (63%), volume resuscitation (61%) and intravenous antibiotics (69%). Acute renal failure (37%) and hepatic dysfunction (17%) were common. The median (inter-quartile range) EO concentration for the study population was 270 (204-411) pmol/L.

Patient characteristics by plasma EO tertile are shown in Table 7.

TABLE 7
Clinical variables according to tertiles of endogenous ouabain.
(1st tertile <118 pmol/L, 2nd tertile 118-207 pmol/L, 3rd tertile >207 pmol/L)

| | 1^{st} Tertile | 2^{nd} Tertile | 3^{rd} Tertile | P value |
|---|---|---|---|---|
| Variable | | | | |
| Age (years) | 57 ± 11 | 59 ± 18 | 57 ± 16 | 0.9 |
| Male | 61% | 56% | 56% | 0.9 |
| African American | 56% | 61% | 50% | 0.6 |
| Mean arterial pressure | 87 ± 22 | 75 ± 19 | 78 ± 21 | 0.2 |
| (mmHg) | | | | |
| Heart rate (bpm) | 97 ± 21 | 97 ± 27 | 98 ± 27 | 0.9 |
| NT-proBNP | 1494 | 7290 | 7853 | <0.001 |
| (pg/mL; Median, IQR) | (392-3497) | (3282-15152) | (3264-19501) | |
| Creatinine (mg/dL) | 1.3 ± 0.8 | 1.9 ± 1.3 | 3.4 ± 2.6 | 0.002 |
| Total bilirubin (mg/dL) | 0.6 ± 0.5 | 1.8 ± 2.3 | 3.9 ± 5.6 | 0.03 |
| Ejection fraction (%) | 56 ± 15 | 47 ± 16 | 55 ± 18.8 | 0.4 |
| Left ventricular hypertrophy | 22% | 21% | 30% | 0.9 |
| APACHE II score | 11.8 ± 7.4 | 14.9 ± 6.2 | 19.9 ± 10.2 | 0.02 |
| Death | 11% | 39% | 44% | 0.07 |
| Acute medical diagnosis | | | | |
| Acute Kidney Injury | 11% | 39% | 61% | 0.008 |
| Acute liver failure | 6% | 0% | 44% | 0.001 |
| Gastrointestinal bleeding | 33% | 11% | 11% | 0.1 |
| Pneumonia | 33% | 50% | 50% | 0.5 |
| Acute respiratory distress | 29% | 44% | 39% | 0.6 |
| syndrome | | | | |
| Sepsis | 33% | 50% | 44% | 0.6 |
| Shock | 11% | 29% | 17% | 0.4 |
| ICU interventions | | | | |
| Mechanical ventilation | 50% | 72% | 67% | 0.4 |
| Intravenous vasopressors | 17% | 33% | 17% | 0.4 |
| Transfusion of blood products | 28% | 28% | 17% | 0.7 |
| Volume resuscitation | 67% | 50% | 67% | 0.5 |
| Antibiotics | 56% | 78% | 72% | 0.4 |
| Steroids | 28% | 28% | 33% | 0.9 |
| Medications prior to ICU | | | | |
| admission | | | | |
| ACEi or ARB | 0% | 17% | 33% | 0.03 |
| β-blocker | 22% | 33% | 56% | 0.1 |
| Diuretic | 6% | 22% | 72% | <0.001 |
| Aldosterone antagonist | 6% | 11% | 22% | 0.3 |
| Bronchodilator | 22% | 22% | 22% | 1.0 |
| Continuous home oxygen | 6% | 11% | 11% | 0.8 |
| Prior Medical History | | | | |
| Coronary artery disease | 11% | 22% | 33% | 0.3 |
| Congestive heart failure | 0% | 11% | 28% | 0.04 |
| Diabetes mellitus | 22% | 39% | 33% | 0.5 |
| Hypertension | 44% | 50% | 72% | 0.2 |
| Chronic obstructive | 11% | 22% | 22% | 0.6 |
| pulmonary disease | | | | |
| Chronic renal disease | 17% | 17% | 22% | 0.8 |

NT-proBNP = amino-terminal B-type natriuretic peptide

Left ventricular systolic function and left ventricular hypertrophy as determined by echocardiography were similar between tertiles of ouabain. Age, gender, race, blood pressure or heart rate also did not vary statistically among tertiles. However, patients in the highest tertile were more likely to have a history of heart failure and to have previously been prescribed diuretics and angiotensin converting enzyme inhibitors or angiotensin receptor blockers.

Severity of illness measured by APACHE score correlated with ouabain concentrations (β=0.354, p=0.009) and increased with each tertile of ouabain (11.8±7.4 vs. 14.9±6.2 vs. 19.9±10.2, p=0.02) as show in FIG. 5A. In-hospital mortality was also highest in those patients with increased ouabain (11% vs. 39% vs. 44%, p=0.07) (FIG. 5C). The prevalence of pneumonia, shock, sepsis, ARDS, or acute ICU interventions did not vary between each tertile of ouabain.

Patients had increasing serum creatinine concentrations (1.3±0.8 mg/dL vs. 1.9±1.3 mg/dL vs. 3.4±2.6 mg/dL, p=0.002, FIG. 5B) and a higher prevalence of acute renal failure (11% vs. 39% vs. 61%, p=0.008) with each incremental tertile of ouabain. Patients with the highest tertile of EO had increased serum total bilirubin concentrations (0.6±0.5 mg/dL vs. 1.8±2.3 mg/dL vs. 3.9±5.6 mg/dL, p=0.03) and a higher prevalence of acute hepatic dysfunction (6% vs. 0% vs. 44%, p=0.001). NT-proBNP also increased with each tertile of EO (median[IQR]: 1494 (392-3497) vs. 7290 (3282-15152) vs. 7853 (3264-19501); p<0.001).

Regression analysis of continuous variables showed that plasma EO concentrations were associated with increased serum creatinine (β=0.497, P<0.001, FIG. 5D), total bilirubin concentrations (β=0.438, P=0.001) and NT-proBNP (β=0.5, P<0.001). All three associations remained independently significant after adjustments for significant covariates (creatinine: β=0.334 P<0.006; bilirubin: β=0.404, P<0.001; NT-proBNP: β=0.3 P<0.013).

In conclusion, EO concentrations are elevated in patients with critical illness. Concentrations are associated with severity of illness, renal function, liver function, and NT-proBNP. While metabolism may play a role in the observed elevations of EO concentrations, EO may also be secreted in response to critical illness. Furthermore, EO might directly cause increased natriuretic peptide levels.

These results on vascular and critical ill patients of plasma EO suggest that plasma EO levels increased in both conditions and may be used as serum biomarker of kidney possible damage.

### Experimental Model

To investigate the in vivo adverse consequences of high EO plasma levels and the gene profile combination the authors studied the effects of ouabain in rats. As already published,^{29,31,32} chronic infusion (8 weeks) of low doses of ouabain in OHR rats significantly raised systolic blood pressure (SBP) (FIG. 3a). In addition, OHR (n=7), as compared to controls (n=7), exhibited a significant increase of plasma creatinine (+14%, p<0.05, FIG. 3b) while urinary creatinine was similar (controls 36.1±0.51, OHR 33.8±1.4 mg/24 h) thus leading to a significant reduction of creatinine clearance in OHR (−18%, p<0.02) (FIG. 4c). Urinary protein excretion was also significantly increased in OHR (+54%, p<0.05) (FIG. 3d). Immunohistochemical analysis of podocyte proteins expression in OHR revealed a significant reduction of staining for nephrin (FIG. 3e), but not for synaptopodin (not shown), as compared to control rats. This data was confirmed by Western blot analysis of renal microsomes where a reduced expression of nephrin, normalized for actin content, but not of synaptopodin (not shown) was detected in OHR but not in control rats (FIG. 3f). The effect of the exposure to ouabain (10⁻⁹ M ouabain for 4 days) on nephrin expression was then evaluated by Western blot analysis in cultured rat glomerular podocytes. A decreased expression of nephrin, normalized for actin (FIG. 3g), but not of synaptopodin (not shown), was confirmed in ouabain-treated podocytes when compared to the control group.

### Discussion

The primary result of this study is the identification of new pre-operative humoral biomarkers of AKI after cardiac surgery. To our knowledge this is the first time that EO and related gene network have been related to AKI and post-operative mortality. EO was found to be the strongest pre-operative biomarker for AKI and in-hospital mortality after correction for confounders as indicates by logistic linear regression analysis (Table 5). EUROSCORE and ACEF, considered two of the strongest prognostic risk stratification criteria, have a lower impact than EO.

Furthermore, the present invention's experimental data indicate that a relatively small but sustained increase in EO plasma concentrations associates with a deterioration of renal function (increase plasma creatinine and reduction of eGFR) and increase in proteinuria. The glomerular damage responsible for these organ alterations resides in the podocytes where ouabain decreases the expression of nephrin, a structural protein involved in the formation of the glomerular filtration barrier via the activation of a complex signaling pathway.³¹ The ouabain-mediated reduction of nephrin protein expression may be responsible for the changes of the glomerular filtration barrier permeability leading to protein leakage. Translated to humans, these data suggest that the power of the baseline EO plasma levels, i.e EO plasma levels before surgery in predicting AKI may be associated to an underlying glomerular damage.

### Genomic Actions on EO

Gene variants thought to affect endogenous ouabain synthesis and transport, rather than EO plasma levels itself, were considered in the present invention. As previously shown for RAAS (Renin Angiotensin Aldosterone System) or other hormonal systems, plasma levels of a hormone do not reflect the paracrine and autocrine changes that occur, for example, with different Na⁺ challenge.³² The same was hypothesized for ouabain.

The rationale for using this particular combination of genes or regulatory gene network, originate from the growing experimental evidences related to EO synthesis, metabolism and activity. By using a top-down approach, the authors of the present invention identified two molecular mechanisms, adducin polymorphisms (ADD1, ADD2, ADD3)²⁹ and endogenous ouabain²⁶ responsible for changes associated to the transition from normotension to hypertension. The mutant β-adducin increase the renal Na⁺/K⁺-ATPase activity, enhance tubular Na⁺ reabsorption and blood pressure.²⁹ Ouabain exerts a biphasic effect on Na⁺/K⁺-ATPase, either stimulating or inhibiting its activity, as function of low (subnanomolar) or high (nanomolar) concentrations. Doubling the subnanomolar ouabain plasma concentrations in rats by ouabain infusion, BP, renal Na⁺/K⁺-ATPase activity and Na⁺/K⁺-ATPase-Src-ERK1/2 signaling increase.²⁶ Gene variants involving ouabain metabolism may modulate this signaling by changing ouabain tissue levels. Recently,³¹ the authors showed in human kidney tissues LSS rs2254524 affected LSS mRNA expression. Analogously, human adrenocortical cells transfected with mutant variant of LSS showed a significant decrease in both LSS mRNA and LSS protein. However, LSS activity and endogenous ouabain level were higher in mutant compared to wild-type transfected cells. The LSS rs914247 used in the GENEO profile resulted in strong linkage disequilibrium with rs2254524 a missense polymorphism (Val642Leu; CC=Val and AA=Leu) located in exon 20 of LSS gene. This means that genotyping data from both these SNPs give the same genetic variation information, that is one SNP is the proxy of the other.

HSD3B1 is involved in ouabain synthesis²⁵ and series of tag SNPs are associated with BP variations.^{25,29} The MDR1 rs1045642 was previously shown to affect the transmembrane transport of cardiacglycoside and the expression of the protein in entherocytes associated to variations of digoxin and ouabain plasma levels.²⁵ Furthermore, prolonged ouabain treatment upregulates the Na⁺ pump a₂-subunit-NCX1-TRPC6 Ca²⁺ signaling pathway in arterial myocytes in vitro as well as in vivo. This may explain the augmented myogenic responses and development of kidney damage in stress condition. Indeed, GENEO profile includes SLC8A1 and TRPC6 SNPs selected with a whole genome scan in salt sensitive hypertension patients.

In respect to hospital mortality, GENEO has a specificity of 71.0% and a negative predictive value of 98.8%, with a sensitivity of 70% but a low positive predictive value due to the small number of event in the present study. These data of sensitivity and specificity referring to a pre-operative circulating biomarker are similar or even better than those recently reported for baseline plasma creatinine and cystatin C in patients admitted in emergency department that may develop AKI.³³

GENEO profile may contribute to the success of personalized medicine since it can possibly identify patients who can benefit from targeted therapy. Recently,³⁴ rostafuroxin, a safe, potent and selective inhibitor of EO and its RGN, has been shown to have antihypertensive activity in patients. If EO truly leads to renal dysfunction, the same pharmacogenomic approach may be used in cardiac surgery patients carrying the GENEO profile and may provide a specific pre-operative therapy.

In conclusion, EO concentrations are elevated in patients with critical illness. Concentrations are associated with both severity of illness and renal function. While metabolism may play a role in the observed elevations of EO concentrations in the pre-operative phase, EO may also be secreted in response to critical illness, and be responsible of kidney damage and in-hospital mortality.

### REFERENCES

1. Manunta P, et al. Biochim Biophys Acta. 1802, 1214-1218 (2010).
2. Ferrari P, et al. J Pharmacol Exp Ther. 285, 83-94 (1998).
3. Ferrandi M, Molinari I, Barassi P et al. J Biol Chem. 279, 33306-33314 (2004).
4. Tripodi G, Citterio L, Kouznetsova T et al. Am J Hypertens. 22, 357-363 (2009).
5. Manunta P, Ferrandi M, Bianchi G, Hamlyn J M. J Hypertens. 27, 9-18 (2009).
6. Pitzalis M V, Hamlyn J M, Messaggio E et al. Eur J Heart Fail. 8, 179-186 (2006).
7. Manunta P, Hamlyn J M, Simonini M et al. J Hypertens. 29, 349-356. (2011).
8. Ferrari P, et al. Med Hypotheses. 68, 1307-1314 (2007).
9. Citterio L, et al. Biochim Biophys Acta. 1802, 1285-1298 (2010).
10. Raina H, et al. Am J Physiol Heart Circ Physiol. 298, H2093-2101.
11. Bauer N, Muller-Ehmsen J, Kramer U et al. Hypertension. 45, 1024-1028 (2005).
12. Cho K C, Himmelfarb J, Paganini E et al. J Am Soc Nephrol. 17, 3132-3138 (2006).
13. Englberger L, Suri R M, Li Z et al. Am J Kidney Dis. 56, 623-631 (2010).
14. Rogues F, et al. Eur J Cardiothorac Surg. 15, 816-822 (1999).
15. Ranucci M, et al. Circulation. 119, 3053-3061 (2009).
16. Coca S G, Yalavarthy R, Concato J, Parikh C R: Kidney Int. 73, 1008-1016 (2008).
17. Haase M, Bellomo R, Haase-Fielitz A J Am Coll Cardiol. 55, 2024-2033 (2010).
18. Haase M, et al., Am J Kidney Dis. 54, 1012-1024 (2009).
19. Huang B S, et al., Am J Physiol Heart Circ Physiol. 299, H422-430 (2010).
20. Fedorova O V, et al. J Hypertens. 28, 361-366 (2010).
21. Pullen M A, at al. J Immunol Methods. 336, 235-241 (2008).
22. Schoner W, Scheiner-Bobis G. Am J Cardiovasc Drugs. 7, 173-189 (2007).
23. Pullen M A, Brooks D P, Edwards R M J Pharmacol Exp Ther. 310, 319-325 (2004).
24. Krep H, Price D A, Soszynski P et al. Am J Hypertens. 8, 921-927 (1995).
25. Ferrandi M, Manunta P, Balzan S et al., Hypertension. 30, 886-896 (1997).
26. De Bonis M, Lapenna E, La Canna Get al., Circulation. 112, 1402-1408 (2005).
27. Bellomo R, et al. Crit Care. 8, R204-212 (2004).
28. Mehta R L, Kellum J A, Shah S V et al., Crit Care. 11, R31 (2007).
29. Doi M, Takahashi Y, Komatsu R et al., Nat Med. 16, 67-74 (2010).
30. Ferrandi M, Cusi D, Molinari I et al., J Mol Med. 88, 203-217 (2010).
31. Jones N, Blasutig 1M, Eremina V et al., Nature. 440, 818-823 (2006).
32. Boddi M, Poggesi L, Coppo M et al. Hypertension. 31, 836-842 (1998).
33. Soto K, Coelho S, Rodrigues B et al., Clin J Am Soc Nephrol. 5, 1745-1754 (2010).
34. Lanzani C, Citterio L, Glorioso N et al., Sci Transl Med. 2, 59ra87 (2010).
35. Bignami E et al., J Steroids Hormon Sci. 2011:in press.
36. Shah KB, et al. Am J Med 2007; 120(12):1071-7.

1. A method for predicting the risk of developing acute kidney injury in a subject from which a biological sample is obtained, said method comprising:
detecting the presence of at least one genomic single nucleotide polymorphism (SNP) selected from the group consisting of:
ADD1 rs4961 Trp [allelic genotype GT or TT],
ADD2 rs4984 [allelic genotype CT or TT],
HSD3B1 rs2236780 [allelic genotype GG],
LSS rs914247 [allelic genotype AA],
MDR1 rs1045642 [allelic genotype TC or CC],
SLC8A1 rs11893826 [allelic genotype AA], and
TRPC6 rs7925662 [allelic genotype CC] from said biological sample,
wherein the presence of said allelic genotype is predictive of the risk of developing acute kidney injury.

2. The method according to claim 1 further comprising measuring the level of endogenous ouabain in a biological sample, wherein a level greater than 207 pM is predictive of the risk of developing acute kidney injury.

3. The method according to claim 1 wherein the presence of at least two genomic single nucleotide polymorphisms SNPs is detected.

4. The method according to claim 3 wherein the two genomic SNPs are:
ADD2 rs4984 [allelic genotype CT or TT] and HSD3B1 rs2236780 [allelic genotype GG];
ADD2 rs4984 [allelic genotype CT or TT] and SLC8A1 rs11893826 [allelic genotype AA];
LSS rs914247 [allelic genotype AA] and SLC8A1 rs11893826 [allelic genotype AA]; or
TRPC6 rs7925662 [allelic genotype CC] and ADD1 rs4961 Trp [allelic genotype GT or TT].

5. The method according to claim 2 wherein the genomic SNP is MDR1 rs1045642 [allelic genotype TC or CC].

6. The method according to claim 1 wherein the biological sample is a biological fluid or a tissue.

7. The method according to claim 2 wherein the level of endogenous ouabain is measured by scintillation proximity assay.

8. The method according to claim 1 to predict the risk of developing acute kidney injury after stress.

9. The method according to claim 8 wherein the stress is an acute hemodynamic stress.

10. The method according to claim 8 wherein the stress is caused by a surgery.

11. The method according to claim 10 wherein the surgery is a cardiac or vascular surgery.

12. A kit for predicting the risk of developing acute kidney injury according to the method of claim 1 comprising reagents to detect at least one single nucleotide polymorphism (SNP) selected from the group consisting of:
ADD1 rs4961 Trp [allelic genotype GT or TT],
ADD2 rs4984 [allelic genotype CT or TT],
HSD3B1 rs2236780 [allelic genotype GG],
LSS rs914247 [allelic genotype AA],
MDR1 rs1045642 [allelic genotype TC or CC],
SLC8A1 rs11893826 [allelic genotype AA], and
TRPC6 rs7925662 [allelic genotype CC].

13. The kit according to claim 12 comprising reagents to detect at least one combination of SNPs selected from the group consisting of:
ADD2 rs4984 [allelic genotype CT or TT] and HSD3B1 rs2236780 [allelic genotype GG];
ADD2 rs4984 [allelic genotype CT or TT] and SLC8A1 rs11893826 [allelic genotype AA];
LSS rs914247 [allelic genotype AA] and SLC8A1 rs11893826 [allelic genotype AA]; and
TRPC6 rs7925662 [allelic genotype CC] and ADD1 rs4961 Tip [allelic genotype GT or TT]

14. The kit according to claim 12 further comprising means to measure endogenous ouabain level.

15. The kit according to claim 14 comprising reagents to detect the SNP MDR1 rs1045642 [allelic genotype TC or CC] and means to measure endogenous ouabain level.

16. A selective ouabain inhibitor for use to decrease the risk of developing AKI in a subject at risk of developing AKI predicted according to the method of claim 1.

17. The selective ouabain inhibitor according to claim 16 being rostafuroxin or digibind.

18. A method for decreasing the risk of developing AKI in a subject comprising:
predicting the risk of developing AKI according to claim 1; and
if the subject is at risk, administering to said subject a selective endogenous ouabain inhibitor.

19. The method according to claim 18 wherein the selective endogenous ouabain inhibitor is rostafuroxin or digibind.


SEQUENCE LISTING (9 sequences)

SEQ ID NO: 1 - DNA, 121 bp, Homo sapiens
  misc_feature (61)..(61): n is G or T
cagcagcgggagaagacaagatggctgaactctggccggggcgacgaagcttccgaggaanggcagaatggaagcagtcccaagtcgaagactaaggtgtggacgaacattacacacgatc

SEQ ID NO: 2 - DNA, 121 bp, Homo sapiens
  misc_feature (61)..(61): n is C or T
ttcagagacaggacaggaacgagagccaggctctggtccggccgtgtgcgagttcttcagngttgccctccacatctggagtaacatattggtaggtgtgttttgatgaaggaaacatgac

SEQ ID NO: 3 - DNA, 121 bp, Homo sapiens
  misc_feature (61)..(61): n is A or G
atggcatagtatgaaagatactgggtgggatttccagagactagattctggccctgacccngaacttgagaagcagccacctcagcctccaggcctcttttcttctcatctagaaaatccc

SEQ ID NO: 4 - DNA, 121 bp, Homo sapiens
  misc_feature (61)..(61): n is A or G
agggggagaggccagggactgctacctgcccagaaggcggcagggaggggaagagcagatnaggaggtatagggtgtgccctgggcaaggcagcaggggtaacgaagctctcagtgactcc

SEQ ID NO: 5 - DNA, 121 bp, Homo sapiens
  misc_feature (61)..(61): n is C or T
cagcattgctgagaacattgcctatggagacaacagccgggtggtgtcacaggaagagatngtgagggcagcaaaggaggccaacatacatgccttcatcgagtcactgcctaatgtaagt

SEQ ID NO: 6 - DNA, 121 bp, Homo sapiens
  misc_feature (61)..(61): n is A or G
aaggaagtcaggtaaaaaaaggaaaagcaagaaaaacaacaaaccactcacctccatagcncactcagtaatgcaatggtagttagcagatccaattaccagctactcagtggcactcaat

SEQ ID NO: 7 - DNA, 121 bp, Homo sapiens
  misc_feature (61)..(61): n is C or T
taccacatggccttatgtggatgtttctctagtcattttggtcatctggggcacattttcnagaaacttcttcatgaagggatcttgggaactatttccctgagttcttgtatgttgttca

SEQ ID NO: 8 - DNA, 25 bp, Artificial Sequence
  synthetic primer
ggtgggatttccagagactagattc

SEQ ID NO: 9 - DNA, 19 bp, Artificial Sequence
  synthetic primer
gctgaggtggctgcttctc